(19) United States
(12) Patent Application Publication
(10) Pub. No.: US 2015/0273041 A1
(43) Pub. Date: Oct. 1, 2015

(54) STAPHYLOCOCCUS AUREUS SDRE CNAB DOMAIN AND ITS USE FOR VACCINATION

(71) Applicant: NOVARTIS AG, Basel (CH)

(72) Inventors: Andrea Guido Oreste Manetti, Siena (IT); Luigi Fiaschi, Sovicille (IT); Marco Becherelli, Lastra a Signa (IT); Prachi Prachi, Allston, MA (US); Massimiliano Biagini, Colle Val D'elsa (IT)

(21) Appl. No.: 14/437,862

(22) PCT Filed: Oct. 29, 2013
(86) PCT No.: PCT/EP2013/072542

§ 371 Date: Apr. 23, 2015

(30) Foreign Application Priority Data
Oct. 29, 2012 (GB) 1219420.5

(51) Int. Cl. A61K 39/085; A61K 45/06; C07K 14/31

(52) U.S. Cl. CPC A61K 39/085; C07K 14/31; A61K 45/06

(57) ABSTRACT

The S. aureus Ser-Asp rich fibrinogen/bone sialoprotein-binding protein contains three CnaB domains, and that the third of these provides significant protection against S. aureus infection. Thus a useful S. aureus vaccine can include a SdrE CnaB domain. Furthermore, the SdrE protein has been shown to be relatively resistant to trypsin digestion, which could be connected to the observation that SdrE contains an isopeptide bond within the third CnaB domain.

This application claims the benefit of UK provisional application 1219420.5 filed Oct. 29, 2012, the complete contents of all of which are hereby incorporated herein by reference for all purposes.

### TECHNICAL FIELD

The invention is in the field of Staphylococcus aureus immunogens.

### BACKGROUND ART

Various vaccines against S. aureus are currently being investigated e.g. see reference 1. One approach, as disclosed in reference 2 uses polypeptides containing a CnaB domain. This domain was first described in a S. aureus collagen-binding surface protein as a region that does not mediate collagen binding. FIG. 28 in reference 2 shows that a CnaB domain from the S. aureus SdrD protein confers protection in a mouse model against infection with strain USA300.

It is an object of the invention to provide further and improved immunogens for eliciting an immune response against S. aureus.

### DISCLOSURE OF THE INVENTION

Reference 2 identifies the SdrD protein of S. aureus as containing a CnaB domain. The inventors have found that the S. aureus Ser-Asp rich fibrinogen/bone sialoprotein-binding protein (SdrE) contains three CnaB domains (‘CnaBE1’, ‘CnaBE2’, and ‘CnaBE3’), and that the third of these domains provides significant protection against S. aureus infection, as demonstrated by a reduction in kidney abscess formation. Moreover, the inventors have shown cross-protection even against strains which do not express SdrE. Furthermore, the SdrE protein has been shown to be relatively resistant to trypsin digestion, which could be connected to the observation that SdrE contains an isopeptide bond within its third CnaB domain (i.e. within CnaBE3).

In a first aspect, the invention provides a polypeptide comprising a SdrE CnaBE3 domain, wherein the polypeptide does not comprise a full-length SdrE protein.

In a second aspect, the invention provides a polypeptide comprising a SdrE CnaBE3 domain, wherein the polypeptide has fewer than 500 amino acids.

In a third aspect, the invention provides a polypeptide comprising a fragment of a S. aureus SdrE protein, wherein: (a) the fragment includes the SdrE CnaBE3 domain; and (b) the polypeptide does not comprise a full-length SdrE protein.

In a fourth aspect, the invention provides a polypeptide comprising a S. aureus CnaB domain, wherein the CnaB domain includes an isopeptide bond. The CnaB domain is preferably CnaBE3.

In a fifth aspect, the invention provides a polypeptide comprising a mutant S. aureus SdrE CnaBE3 domain wherein, at one or more amino acid position(s) where the native S. aureus SdrE CnaBE3 domain has an asparagine residue, the mutant has either (i) an amino acid deletion or (ii) an amino acid substitution.

Similarly, the invention provides a polypeptide comprising a mutant S. aureus SdrE CnaBE3 domain wherein, at one or more amino acid position(s) where the native S. aureus SdrE CnaBE3 domain has an aspartate residue, the mutant has either (i) an amino acid deletion or (ii) an amino acid substitution.

Similarly, the invention provides a polypeptide comprising a mutant S. aureus SdrE CnaBE3 domain wherein, at one or more amino acid position(s) where the native S. aureus SdrE CnaBE3 domain has a lysine residue, the mutant has either (i) an amino acid deletion or (ii) an amino acid substitution.

In a sixth aspect, the invention provides a polypeptide comprising at least two CnaB domains, wherein: either (a) at least one of the CnaB domains is a CnaBE3 domain and at least one CnaB domain is not a SdrE CnaB domain; or (b) the polypeptide comprises at least two CnaBE3 domains. Such polypeptides can comprise amino acid sequence A(LB)ₙ as disclosed in reference 2 (e.g. see pages 9-13 therein), provided that at least one A and/or B is a CnaBE3 domain.

These polypeptides of the invention are useful as components of immunogenic compositions for raising immune responses e.g. to protect against S. aureus infection.

### SdrE

The S. aureus SdrE protein is a Ser-Asp rich fibrinogen/bone sialoprotein-binding protein, as discussed in more detail in references 3-8. In S. aureus bacteria it is anchored in the cell wall. In the Newman NWMN_{—}0525 strain its amino acid sequence is SEQ ID NO: 1:

MINRDNKKAITKKGMISNRLNKFSIRKYTVGTASILVGTTLIFGLGNQEA
KAAENTSTENAKQDDATTSDNKEVVSETENNSTTENNSTNPIKKETNTDS
QPEAKKESTSSSTQKQQNNVTATTETKPQNIEKENVKPSTDKTATEDTSV
ILEEKKAPNNTNNDVTTKPSTSEPSTSEIQTKPTTPQESTNIENSQPQPT
PSKVDNQVTDATNPKEPVNVSKEELKNNPEKLKELVRNDSNTDHSTKPVA
TAPTSVAPKRVNAKMRFAVAQPAAVASNNVNDLIKVTKQTIKVGDGKDNV
AAAHDGKDIEYDTEFTIDNKVKKGDTMTINYDKNVIPSDLTDKNDPIDIT
DPSGEVIAKGTFDKATKQITYTFTDYVDKYEDIKSRLTLYSYIDKKTVPN
ETSLNLTFATAGKETSQNVTVDYQDPMVHGDSNIQSIFTKLDEDKQTIEQ
QIYVNPLKKSATNTKVDIAGSQVDDYGNIKLGNGSTIIDQNTEIKVYKVN
SDQQLPQSNRIYDFSQYEDVTSQFDNKKSFSNNVATLDFGDINSAYIIKV
VSKYTPTSDGELDIAQGTSMRTTDKYGYYNYAGYSNFIVTSNDTGGGDGT
VKPEEKLYKIGDYVWEDVDKDGVQGTDSKEKPMANVLVTLTYPDGTTKSV
RTDANGHYEFGGLKDGETYTVKFETPTGYLPTKVNGTTDGEKDSNGSSVT
VKINGKDDMSLDTGFYKEPKYNLGDYVWEDTNKDGIQDANEPGIKDVKVT
LKDSTGKVIGTTTTDASGKYKFTDLDNGNYTVEFETPAGYTPTVKNTTAD
DKDSNGLTTTGVIKDADNMTLDRGFYKTPKYSLGDYVWYDSNKDGKQDST
EKGIKDVTVTLQNEKGEVIGTTKTDENGKYRFDNLDSGKYKVIFEKPAGL
TQTVTNTTEDDKDADGGEVDVTITDHDDFTLDNGYFEEDTSDSDSDSDSD
SDSDSDSDSDSDSDSDSDSDSDSDSDSDSDSDSDSDSDSDSDSDSDSDSD
SDSDSDSDSDSDSDSDSDSDSDSDSDSDSDSDSDSDSDSDSDSDSDSDSD
SDSDSDSDSDSDSDSDSDSDSDSDSDSDSDSDSDSDSDSDSDSDSDSDSD
SDSDSDAGKHTPVKPMSTTKDHHNKAKALPETGSENNGSNNATLFGGLFA
ALGSLLLFGRRKKQNK

The SdrE sequence from many more strains is known in the art. A search of the NCBI polypeptide sequence database for SdrE sequences in S. aureus at the time of filing reveals 73 hits, and a BLINK search using SEQ ID NO: 1 gives the SdrE sequence for at least strains COL (sequence accession number AAW37719), ATCC BAA-39 (EFM05571), CIG1612 (EHT61800), CIG2018 (EHT70059), USA300_TCH1516 (ABX28583), USA300_FPR3757 (ABD22410), CIG547 (EHT48726), CIGC345D (EHT90441), 21340 (EHM84415), CIG1770 (EHT65828), CIG1114 (EHT20837), TW20 (CBI48512), 21272 (EHP00675), A8819 (EFG44197), ATCC 51811 (EFH25573), Newbould 305 (EJE56337), JKD6008 (ADL64631), T0131 (AEB87697), etc.

The BLINK hits have between 1131-1166 amino acids, with most of this length variation arising from differences in the lengths of the Ser-Asp repeats. Aside from this variation, and the presence or absence of a 5-mer PSTSE sequence (SEQ ID NO: 44), the sequence is otherwise very highly conserved between many strains. Thus the nascent sequence may generally be represented as follows:

[SEQ ID NO: 2]-X¹-[SEQ ID NO: 3]-X²-[SEQ ID NO: 4] (formula ‘A’)

where:
SEQ ID NO: 2 is:

MINRDNKKAITKKGMISNRLNKFSIRKYTVGTASILVGTTLIFGLGNQEA
KAAENTSTENAKQDDATTSDNKEVVSETENNSTTENNSTNPIKKETNTDS
QPEAKKESTSSSTQKQQNNVTATTETKPQNIEKENVKPSTDKTATEDTSV
ILEEKKAPNNTNNDVTTK

SEQ ID NO: 3 is:

PSTSEIQTKPTTPQESTNIENSQPQPTPSKVDNQVTDATNPKEPVNVSKE
ELKNNPEKLKELVRNDSNTDHSTKPVATAPTSVAPKRVNAKMRFAVAQPA
AVASNNVNDLIKVTKQTIKVGDGKDNVAAAHDGKDIEYDTEFTIDNKVKK
GDTMTINYDKNVIPSDLTDKNDPIDITDPSGEVIAKGTFDKATKQITYTF
TDYVDKYEDIKSRLTLYSYIDKKTVPNETSLNLTFATAGKETSQNVTVDY
QDPMVHGDSNIQSIFTKLDEDKQTIEQQIYVNPLKKSATNTKVDIAGSQV
DDYGNIKLGNGSTIIDQNTEIKVYKVNSDQQLPQSNRIYDFSQYEDVTSQ
FDNKKSFSNNVATLDFGDINSAYIIKVVSKYTPTSDGELDIAQGTSMRTT
DKYGYYNYAGYSNFIVTSNDTGGGDGTVKPEEKLYKIGDYVWEDVDKDGV
QGTDSKEKPMANVLVTLTYPDGTTKSVRTDANGHYEFGGLKDGETYTVKF
ETPTGYLPTKVNGTTDGEKDSNGSSVTVKINGKDDMSLDTGFYKEPKYNL
GDYVWEDTNKDGIQDANEPGIKDVKVTLKDSTGKVIGTTTTDASGKYKFT
DLDNGNYTVEFETPAGYTPTVKNTTADDKDSNGLTTTGVIKDADNMTLDR
GFYKTPKYSLGDYVWYDSNKDGKQDSTEKGIKDVTVTLQNEKGEVIGTTK
TDENGKYRFDNLDSGKYKVIFEKPAGLTQTVTNTTEDDKDADGGEVDVTI
TDHDDFTLDNGYFEEDT

SEQ ID NO: 4 is:

AGKHTPVKPMSTTKDHHNKAKALPETGSENNGSNNATLFGGLFAALGSLL
LFGRRKKQNK

X¹ is an optional PSTSE sequence (SEQ ID NO: 44), and
X² is between 20-250 amino acids long and is either (i) multiple repeats of SD or (ii) a mixture of both SD and AD sequences.

Thus SEQ ID NO: 1 is an example of formula ‘A’, wherein X¹ is present and X² is 83 repeats of SD.

The invention can use any of these known SdrE sequences. In general, a SdrE used with the invention will comprise a sequence having at least 90% identity to SEQ ID NO: 3 (e.g. ≧91% identity, ≧92% identity, ≧93% identity, ≧94% identity, ≧95% identity, ≧96% identity, ≧97% identity, ≧98% identity, ≧99% identity, or 100% identity) and will, when administered to a human or mouse, elicit antibodies which recognise the wild-type S. aureus protein which is expressed as SEQ ID NO: 1.

Where an embodiment of the invention utilises a fragment of a S. aureus SdrE protein, that fragment will generally be a fragment of a sequence having at least 90% identity to SEQ ID NO: 3 (e.g. ≧91% identity, ≧92% identity, ≧93% identity, ≧94% identity, ≧95% identity, ≧96% identity, ≧97% identity, ≧98% identity, ≧99% identity, or 100% identity). A polypeptide comprising the fragment will, when administered to a human or mouse, elicit antibodies which recognise the wild-type S. aureus protein which is expressed as SEQ ID NO: 1.

One useful fragment of S. aureus SdrE includes a CnaBE3 domain (see below) but includes fewer than 20 Ser-Asp repeats.

Where an embodiment of the invention does not utilise a full-length SdrE protein, it does not utilise a protein having formula ‘A’.

Also, a polypeptide of the invention usually will not comprise an amino acid sequence of formula ‘B’, wherein formula ‘B’ is:

[SEQ ID NO: 5]-X¹-[SEQ ID NO: 3]-X²-[SEQ ID NO: 6]

where:
SEQ ID NO: 5 is:

AENTSTENAKQDDATTSDNKEVVSETENNSTTENNSTNPIKKETNTDSQP
EAKKESTSSSTQKQQNNVTATTETKPQNIEKENVKPSTDKTATEDTSVIL
EEKKAPNNTNNDVTTK

SEQ ID NO: 3 is:

PSTSEIQTKPTTPQESTNIENSQPQPTPSKVDNQVTDATNPKEPVNVSKE
ELKNNPEKLKELVRNDSNTDHSTKPVATAPTSVAPKRVNAKMRFAVAQPA
AVASNNVNDLIKVTKQTIKVGDGKDNVAAAHDGKDIEYDTEFTIDNKVKK
GDTMTINYDKNVIPSDLTDKNDPIDITDPSGEVIAKGTFDKATKQITYTF
TDYVDKYEDIKSRLTLYSYIDKKTVPNETSLNLTFATAGKETSQNVTVDY
QDPMVHGDSNIQSIFTKLDEDKQTIEQQIYVNPLKKSATNTKVDIAGSQV
DDYGNIKLGNGSTIIDQNTEIKVYKVNSDQQLPQSNRIYDFSQYEDVTSQ
FDNKKSFSNNVATLDFGDINSAYIIKVVSKYTPTSDGELDIAQGTSMRTT
DKYGYYNYAGYSNFIVTSNDTGGGDGTVKPEEKLYKIGDYVWEDVDKDGV
QGTDSKEKPMANVLVTLTYPDGTTKSVRTDANGHYEFGGLKDGETYTVKF
ETPTGYLPTKVNGTTDGEKDSNGSSVTVKINGKDDMSLDTGFYKEPKYNL
GDYVWEDTNKDGIQDANEPGIKDVKVTLKDSTGKVIGTTTTDASGKYKFT
DLDNGNYTVEFETPAGYTPTVKNTTADDKDSNGLTTTGVIKDADNMTLDR
GFYKTPKYSLGDYVWYDSNKDGKQDSTEKGIKDVTVTLQNEKGEVIGTTK
TDENGKYRFDNLDSGKYKVIFEKPAGLTQTVTNTTEDDKDADGGEVDVTI
TDHDDFTLDNGYFEEDT

SEQ ID NO: 6 is:

AGKHTPVKPMSTTKDHHNKAKA

X¹ is an optional PSTSE sequence (SEQ ID NO: 44) (preferably present), and
X² is between 20-250 amino acids long and is either (i) multiple repeats of SD or (ii) a mixture of both SD and AD sequences (and wherein a preferred X² is a 166-mer consisting of 83 repeats of SD).

Thus a preferred example of formula ‘B’ is SEQ ID NO: 7, a 1076-mer:

AENTSTENAKQDDATTSDNKEVVSETENNSTTENNSTNPIKKETNTDSQP
EAKKESTSSSTQKQQNNVTATTETKPQNIEKENVKPSTDKTATEDTSVIL
EEKKAPNNTNNDVTTKPSTSEPSTSEIQTKPTTPQESTNIENSQPQPTPS
KVDNQVTDATNPKEPVNVSKEELKNNPEKLKELVRNDSNTDHSTKPVATA
PTSVAPKRVNAKMRFAVAQPAAVASNNVNDLIKVTKQTIKVGDGKDNVAA
AHDGKDIEYDTEFTIDNKVKKGDTMTINYDKNVIPSDLTDKNDPIDITDP
SGEVIAKGTFDKATKQITYTFTDYVDKYEDIKSRLTLYSYIDKKTVPNET
SLNLTFATAGKETSQNVTVDYQDPMVHGDSNIQSIFTKLDEDKQTIEQQI
YVNPLKKSATNTKVDIAGSQVDDYGNIKLGNGSTIIDQNTEIKVYKVNSD
QQLPQSNRIYDFSQYEDVTSQFDNKKSFSNNVATLDFGDINSAYIIKVVS
KYTPTSDGELDIAQGTSMRTTDKYGYYNYAGYSNFIVTSNDTGGGDGTVK
PEEKLYKIGDYVWEDVDKDGVQGTDSKEKPMANVLVTLTYPDGTTKSVRT
DANGHYEFGGLKDGETYTVKFETPTGYLPTKVNGTTDGEKDSNGSSVTVK
INGKDDMSLDTGFYKEPKYNLGDYVWEDTNKDGIQDANEPGIKDVKVTLK
DSTGKVIGTTTTDASGKYKFTDLDNGNYTVEFETPAGYTPTVKNTTADDK
DSNGLTTTGVIKDADNMTLDRGFYKTPKYSLGDYVWYDSNKDGKQDSTEK
GIKDVTVTLQNEKGEVIGTTKTDENGKYRFDNLDSGKYKVIFEKPAGLTQ
TVTNTTEDDKDADGGEVDVTITDHDDFTLDNGYFEEDTSDSDSDSDSDSD
SDSDSDSDSDSDSDSDSDSDSDSDSDSDSDSDSDSDSDSDSDSDSDSDSD
SDSDSDSDSDSDSDSDSDSDSDSDSDSDSDSDSDSDSDSDSDSDSDSDSD
SDSDSDSDSDSDSDSDSDSDSDSDSDSDSDSDSDSDSDSDSDSDSDSDSD
SDSDAGKHTPVKPMSTTKDHHNKAKA

and so a polypeptide of the invention usually will not comprise SEQ ID NO: 7.

### CnaB Domains

The CnaB domain is a well-recognised protein structure [9] having a prealbumin-like beta-sandwich fold of seven strands in two sheets with a Greek key topology. The SCOP database [10] includes “Cna protein B-type domain” as both a family (49479) and a super-family (49478). In the Pfam database [11] the CnaB domain is entry PF05738. Although the CnaB domain is defined on the basis of secondary protein structure, this structure stems from patterns of amino acids which are readily analysed, and the presence of a CnaB domain can be predicted with relative ease merely on the basis of amino acid sequence, and they are readily identified by conserved domain searching e.g. using the CDD (Conserved Domain Database) as reported in reference 12.

Examples of CnaB domains are disclosed in reference 2, in various bacterial species. The invention concerns S. aureus proteins which include CnaB domains. There are several examples of such proteins in S. aureus, including the prototypic CNA collagen adhesin (which is typically excluded as an embodiment of the invention), but the main focus of the invention is the Sdr proteins (the Ser-Asp rich proteins, such as SdrA, B, C, D, E and/or F), and in particular SdrE.

S. aureus SdrE is discussed above. It contains three CnaB domains, identified in reference 3 as “B repeats”. The boundaries of the three CnaB domains are shown in FIG. 3 of reference 3 based on the Newman strain, and the 3rd CnaB domain (‘CnaBE3’) in SEQ ID NO: 1 is as follows (SEQ ID NO: 8):

KYSLGDYVWYDSNKDGKQDSTEKGIKDVTVTLQNEKGEVIGTTKTDENGK
YRFDNLDSGKYKVIFEKPAGLTQTVTNTTEDDKDADGGEVDVTITDHDDF
TLDNGYFEEDT

Using an alignment of SEQ ID NO: 1 with the SdrE sequence from any other S. aureus strain, SEQ ID NO: 8 permits the CnaBE3 domain to be readily located in that other strain. The invention can use a CnaBE3 domain from any such strain, although the Newman strain is preferred.

In general, therefore, a CnaBE3 domain utilised with the invention will have at least 95% identity to SEQ ID NO: 8 (e.g. ≧96% identity, ≧97% identity, ≧98% identity, ≧99% identity, or 100% identity) and will, when administered to a human or mouse, elicit antibodies which recognise an epitope which (i) is within SEQ ID NO: 8 or (ii) includes amino acids within SEQ ID NO: 8. In other words, the CnaBE3 domain will elicit antibodies which cross-react with the wild-type CnaBE3 domain identified above. In some embodiments the epitope is within SEQ ID NO: 27 or includes amino acids within SEQ ID NO: 27.

The CnaBE3 domain of SEQ ID NO: 8 is a 111-mer, thus representing about 9.5% of the total SdrE protein. Thus a polypeptide of the invention which comprises a CnaBE3 domain can be substantially shorter than a full-length SdrE protein. A CnaBE3-containing polypeptide of the invention can thus have fewer than 500 amino acids e.g. fewer than 400aa, fewer than 350aa, fewer than 300aa, fewer than 250aa, fewer than 200aa, or fewer than 150aa.

When a polypeptide of the invention includes a CnaBE3 domain, any amino acids upstream and/or downstream of the domain can be the same as the upstream/downstream residues in a S. aureus SdrE protein, or they can be different. For instance, when the polypeptide comprises a sequence {A}-{B}-{C} where {B} is a CnaBE3 domain: (a) the C-terminus of {A} can be the same as or different from residues 102-111 of SEQ ID NO: 1; and/or (b) the N-terminus of {C} can be the same as or different from residues 941-951 of SEQ ID NO: 1. Thus the CnaBE3 domain of {B} can be taken as a specific fragment from SdrE, or it can included as part of a larger fragment from SdrE. Where sequence {C} is present, this ideally includes fewer than 20 Ser-Asp repeats e.g. fewer than 10, fewer than 5, or even zero. In one useful embodiment, sequence {A} does include a short portion of the corresponding region within the CnaBE2 domain e.g. up to 20 amino acids. For instance, one useful sequence (SEQ ID NO: 27) retains the final 15 amino acids from CnaBE2, to give a 126-mer fragment of SEQ ID NO: 1:

DADNMTLDRGFYKTPKYSLGDYVWYDSNKDGKQDSTEKGIKDVTVTLQNE
KGEVIGTTKTDENGKYRFDNLDSGKYKVIFEKPAGLTQTVTNTTEDDKDA
DGGEVDVTITDHDDFTLDNGYFEEDT

Each CnaB domain from SdrE includes an EF hand loops which can provide for high affinity binding of calcium. Thus a polypeptide of the invention can include Ca⁺⁺ within a CnaB domain.

The CnaBE3 domain is well downstream of the ‘SdrE₅₃₋₆₃₂’ protein disclosed in reference 1.

SEQ ID NO: 8 has sequence identity to the five CnaB domains from SdrD disclosed as SEQ ID NOs: 134-138 in reference 2 (calculated by CLUSTALW):

| | 134 | 135 | 136 | 137 | 138 |
|---|---|---|---|---|---|
| SEQ ID NO: 8 | 20% | 40% | 45% | 41% | 95% |

Similarly: when SEQ ID NO: 8 is aligned against the corresponding region in SdrD (e.g. for the Newman strains, against amino acids 1013-1123 of SdrD) it has 94.6% identity, with 6 amino acid differences; and when SEQ ID NO: 8 is aligned against the corresponding region in SdrC (e.g. against amino acids 607-717 of SdrC in ref. 1) it also has 94.6% identity, again with 6 amino acid differences.

### Isopeptide Bonds and Mutant CnaBE3 Domains

A CnaB domain utilised with the invention (and in particular a CnaBE3 domain) can usefully include an isopeptide bond i.e. a bond between the side chains of two amino acids, or between the side chain of one amino acid and a free terminus of a peptide chain. Typically this forms between an amino group in one side chain and a carboxyl or carboxamide group on another side chain e.g. between the amino group on a Lys and a carboxamide on a Gln or Asn, or between the amino group on a Lys and a carboxyl on a Glu or Asp. The two amino acids forming the isopeptide bond will usually both be in the same CnaBE3 domain.

In some embodiments, however, a CnaB domain (and in particular a CnaBE3 domain) is mutated to remove a wild-type asparagine and/or lysine residue, thereby disrupting the formation of an isopeptide bond. In such mutant CnaB domains, at one or more amino acid position(s) where the native domain has an asparagine/lysine residue, the mutant has either (i) an amino acid deletion or (ii) an amino acid substitution. SEQ ID NOs: 9 to 14 are examples of CnaBE3 domains in which wild-type Asn residues are mutated, where ‘X’ is not ‘N’ (and ideally is not ‘N’, ‘Q’, ‘D’ or ‘E’):

9:
KYSLGDYVWYDSXKDGKQDSTEKGIKDVTVTLQNEKGEVIGTTKTDENGK
YRFDNLDSGKYKVIFEKPAGLTQTVTNTTEDDKDADGGEVDVTITDHDDF
TLDNGYFEEDT
10:
KYSLGDYVWYDSNKDGKQDSTEKGIKDVTVTLQXEKGEVIGTTKTDENGK
YRFDNLDSGKYKVIFEKPAGLTQTVTNTTEDDKDADGGEVDVTITDHDDF
TLDNGYFEEDT
11:
KYSLGDYVWYDSNKDGKQDSTEKGIKDVTVTLQNEKGEVIGTTKTDEXGK
YRFDNLDSGKYKVIFEKPAGLTQTVTNTTEDDKDADGGEVDVTITDHDDF
TLDNGYFEEDT
12:
KYSLGDYVWYDSNKDGKQDSTEKGIKDVTVTLQNEKGEVIGTTKTDENGK
YRFDXLDSGKYKVIFEKPAGLTQTVTNTTEDDKDADGGEVDVTITDHDDF
TLDNGYFEEDT
13:
KYSLGDYVWYDSNKDGKQDSTEKGIKDVTVTLQNEKGEVIGTTKTDENGK
YRFDNLDSGKYKVIFEKPAGLTQTVTXTTEDDKDADGGEVDVTITDHDDF
TLDNGYFEEDT
14:
KYSLGDYVWYDSNKDGKQDSTEKGIKDVTVTLQNEKGEVIGTTKTDENGK
YRFDNLDSGKYKVIFEKPAGLTQTVTNTTEDDKDADGGEVDVTITDHDDF
TLDXGYFEEDT

Similarly, SEQ ID NOs: 15 to 26 are examples of CnaBE3 domains in which wild-type Lys residues are mutated, where ‘X’ is not ‘K’:

15:
XYSLGDYVWYDSNKDGKQDSTEKGIKDVTVTLQNEKGEVIGTTKTDENGK
YRFDNLDSGKYKVIFEKPAGLTQTVTNTTEDDKDADGGEVDVTITDHDDF
TLDNGYFEEDT
16:
KYSLGDYVWYDSNXDGKQDSTEKGIKDVTVTLQNEKGEVIGTTKTDENGK
YRFDNLDSGKYKVIFEKPAGLTQTVTNTTEDDKDADGGEVDVTITDHDDF
TLDNGYFEEDT
17:
KYSLGDYVWYDSNKDGXQDSTEKGIKDVTVTLQNEKGEVIGTTKTDENGK
YRFDNLDSGKYKVIFEKPAGLTQTVTNTTEDDKDADGGEVDVTITDHDDF
TLDNGYFEEDT
18:
KYSLGDYVWYDSNKDGKQDSTEXGIKDVTVTLQNEKGEVIGTTKTDENGK
YRFDNLDSGKYKVIFEKPAGLTQTVTNTTEDDKDADGGEVDVTITDHDDF
TLDNGYFEEDT
19:
KYSLGDYVWYDSNKDGKQDSTEKGIXDVTVTLQNEKGEVIGTTKTDENGK
YRFDNLDSGKYKVIFEKPAGLTQTVTNTTEDDKDADGGEVDVTITDHDDF
TLDNGYFEEDT
20:
KYSLGDYVWYDSNKDGKQDSTEKGIKDVTVTLQNEXGEVIGTTKTDENGK
YRFDNLDSGKYKVIFEKPAGLTQTVTNTTEDDKDADGGEVDVTITDHDDF
TLDNGYFEEDT
21:
KYSLGDYVWYDSNKDGKQDSTEKGIKDVTVTLQNEKGEVIGTTXTDENGK
YRFDNLDSGKYKVIFEKPAGLTQTVTNTTEDDKDADGGEVDVTITDHDDF
TLDNGYFEEDT
22:
KYSLGDYVWYDSNKDGKQDSTEKGIKDVTVTLQNEKGEVIGTTKTDENGX
YRFDNLDSGKYKVIFEKPAGLTQTVTNTTEDDKDADGGEVDVTITDHDDF
TLDNGYFEEDT
23:
KYSLGDYVWYDSNKDGKQDSTEKGIKDVTVTLQNEKGEVIGTTKTDENGK
YRFDNLDSGXYKVIFEKPAGLTQTVTNTTEDDKDADGGEVDVTITDHDDF
TLDNGYFEEDT
24:
KYSLGDYVWYDSNKDGKQDSTEKGIKDVTVTLQNEKGEVIGTTKTDENGK
YRFDNLDSGKYXVIFEKPAGLTQTVTNTTEDDKDADGGEVDVTITDHDDF
TLDNGYFEEDT
25:
KYSLGDYVWYDSNKDGKQDSTEKGIKDVTVTLQNEKGEVIGTTKTDENGK
YRFDNLDSGKYKVIFEXPAGLTQTVTNTTEDDKDADGGEVDVTITDHDDF
TLDNGYFEEDT
26:
KYSLGDYVWYDSNKDGKQDSTEKGIKDVTVTLQNEKGEVIGTTKTDENGK
YRFDNLDSGKYKVIFEKPAGLTQTVTNTTEDDXDADGGEVDVTITDHDDF
TLDNGYFEEDT

These mutants may resist formation of isopeptide bonds.

In some embodiments of the invention, however, native lysine and/or asparagine and/or aspartate residues are retained so that isopeptide bond formation is maintained. In particular, it is useful to retain the asparagine at the position corresponding to Asn-104 within SEQ ID NO: 8 (ie. the underlined position in SEQ ID NO: 14).

Combinations with S. aureus Saccharides

Polypeptides of the invention may be used in combination with conjugated S. aureus saccharide antigens. Thus the invention provides an immunogenic composition comprising a combination of: (1) a polypeptide of the invention; and (2) one or more conjugates of a S. aureus exopolysaccharide and a carrier protein.

A conjugate used in component (2) of this combination includes a saccharide moiety and a carrier moiety. The saccharide moiety is from the exopolysaccharide of S. aureus, which is a poly-N-acetylglucosamine (PNAG). The saccharide may be a polysaccharide having the size that arises during purification of the exopolysaccharide from bacteria, or it may be an oligosaccharide achieved by fragmentation of such a polysaccharide e.g. size can vary from over 400 kDa to between 75 and 400 kDa, or between 10 and 75 kDa, or up to 30 repeat units. The saccharide moiety can have various degrees of N-acetylation and, as described in reference 13, the PNAG may be less than 40% N-acetylated (e.g. less than 35, 30, 20, 15, 10 or 5% N-acetylated; deacetylated PNAG is also known as dPNAG). Deacetylated epitopes of PNAG can elicit antibodies that are capable of mediating opsonic killing. The PNAG may or may not be 0-succinylated e.g. it may be 0-succinylated on fewer less than 25, 20, 15, 10, 5, 2, 1 or 0.1% of residues.

The invention also provides an immunogenic composition comprising a combination of: (1) a polypeptide of the invention; and (2) one or more conjugates of a S. aureus capsular saccharide and a carrier protein.

A conjugate used in component (2) of this combination includes a saccharide moiety and a carrier moiety. The saccharide moiety is from the capsular saccharide of a S. aureus. The saccharide may be a polysaccharide having the size that arises during purification of capsular polysaccharide from bacteria, or it may be an oligosaccharide achieved by fragmentation of such a polysaccharide. Capsular saccharides may be obtained from any suitable strain of S. aureus (or any bacterium having a similar or identical saccharide), such as from a type 5 and/or a type 8 S. aureus strain and/or a type 336 S. aureus strain. Most strains of infectious S. aureus contain either Type 5 or Type 8 capsular saccharides. Both have FucNAcp in their repeat unit as well as ManNAcA which can be used to introduce a sulfhydryl group for linkage. The repeating unit of the Type 5 saccharide is →4)-β-D-Man NAcA-(1→4)-α-L-FucNAc(3OAc)-(1→3)-β-D-FucNAc-(1→, whereas the repeating unit of the Type 8 saccharide is →3)-β-D-ManNAcA(4OAc)-(1→3)-α-L-FucNAc(1→3)-α-D-FucNAc(1→. The type 336 saccharide is a β-linked hexosamine with no O-acetylation [14,15] and is cross-reactive with antibodies raised against the 336 strain (ATCC 55804). A combination of a type 5 and a type 8 saccharide is typical, and a type 336 saccharide may be added to this pairing [16].

The carrier moiety in these conjugates will usually be a protein, but usually not one of the antigens of (1). Typical carrier proteins are bacterial toxins, such as diphtheria or tetanus toxins, or toxoids or mutants or fragments thereof. The CRM197 diphtheria toxin mutant [17] is useful. Other suitable carrier proteins include the N. meningitidis outer membrane protein complex [18], synthetic peptides [19,20], heat shock proteins [21,22], pertussis proteins [23,24], cytokines [25], lymphokines [25], hormones [25], growth factors [25], artificial proteins comprising multiple human CD4⁺ T cell epitopes from various pathogen-derived antigens [26] such as N19 [27], protein D from H. influenzae [28-30], pneumolysin [31] or its non-toxic derivatives [32], pneumococcal surface protein PspA [33], iron-uptake proteins [34], toxin A or B from C. difficile [35], recombinant P. aeruginosa exoprotein A (rEPA) [36], etc. In some embodiments the carrier protein is a S. aureus protein, such as an antigen selected from the first, second, third or fourth antigen groups.

Where a composition includes more than one conjugate, each conjugate may use the same carrier protein or a different carrier protein.

Conjugates may have excess carrier (w/w) or excess saccharide (w/w). In some embodiments, a conjugate may include substantially equal weights of each.

The carrier molecule may be covalently conjugated to the carrier directly or via a linker. Direct linkages to the protein may be achieved by, for instance, reductive amination between the saccharide and the carrier, as described in, for example, references 37 and 38. The saccharide may first need to be activated e.g. by oxidation. Linkages via a linker group may be made using any known procedure, for example, the procedures described in references 39 and 40. A preferred type of linkage is an adipic acid linker, which may be formed by coupling a free —NH₂ group (e.g. introduced to a glucan by amination) with adipic acid (using, for example, diimide activation), and then coupling a protein to the resulting saccharide-adipic acid intermediate [41,42]. Another preferred type of linkage is a carbonyl linker, which may be formed by reaction of a free hydroxyl group of a saccharide CDI [43, 44] followed by reaction with a protein to form a carbamate linkage. Other linkers include β-propionamido [45], nitrophenyl-ethylamine [46], haloacyl halides [47], glycosidic linkages [48], 6-aminocaproic acid [49], ADH [50], C₄ to C₁₂ moieties [51], etc. Carbodiimide condensation can also be used [52].

PNAG conjugates may be prepared in various ways e.g. by a process comprising: a) activating the PNAG by adding a linker comprising a maleimide group to form an activated PNAG; b) activating the carrier protein by adding a linker comprising a sulphydryl group to form an activated carrier protein; and c) reacting the activated PNAG and the activated carrier protein to form a PNAG-carrier protein conjugate; or by a process comprising a) activating the PNAG by adding a linker comprising a sulphydryl group to form an activated PNAG; b) activating the carrier protein by adding a linker comprising a maleimide group to form an activated carrier protein; and c) reacting the activated PNAG and the activated carrier protein to form a PNAG-carrier protein conjugate; or by a process comprising a) activating the PNAG by adding a linker comprising a sulphydryl group to form an activated PNAG; b) activating the carrier protein by adding a linker comprising a sulphydryl group to form an activated carrier protein; and c) reacting the activated PNAG and the activated carrier protein to form a PNAG-carrier protein conjugate.

The polypeptides of the invention may be used as carrier proteins for a S. aureus saccharide, to form a covalent conjugate. Thus the invention provides an immunogenic composition comprising a conjugate of (1) a polypeptide of the invention and (2) a S. aureus exopolysaccharide or a S. aureus capsular saccharide. Further characteristics of such conjugates are described above.

Combinations with S. aureus Polypeptide Antigens

The polypeptides of the invention may be used in combination with other (non-SdrE) S. aureus polypeptide antigens. For instance, an immunogenic composition can comprise a polypeptide of the invention in combination with any of the S. aureus antigens disclosed in reference 1, such as one or more of the following antigens, as defined in reference 1: (1) a clfA antigen; (2) a clfB antigen; (3) a esxA antigen; (4) a esxB antigen; (5) a Hla antigen; (6) a isdA antigen; (7) a isdB antigen; (8) a isdC antigen; (9) a isdG antigen; (10) a isdH antigen; (11) a isdI antigen; (12) a sasF antigen; (13) a sdrC antigen; (14) a sdrD antigen; (15) a spa antigen; (16) a sta006 antigen; and/or (17) a sta011 antigen. In one embodiment, the invention provides an immunogenic composition which comprises a polypeptide of the invention, comprising a CnaBE3 domain, in combination with one or more of: (a) a mutant hemolysin; (b) a sta006 antigen; (c) a sta011 antigen; (d) an EsxA antigen; and/or (e) an EsxB antigen.

The S. aureus hemolysin (‘Hla’) is also known as ‘alpha toxin’. In the NCTC 8325 strain Hla has amino acid sequence SEQ ID NO: 28 (GI:88194865):

MKTRIVSSVTTTLLLGSILMNPVANAADSDINIKTGTTDIGSNTTVKTGD
LVTYDKENGMHKKVFYSFIDDKNHNKKLLVIRTKGTIAGQYRVYSEEGAN
KSGLAWPSAFKVQLQLPDNEVAQISDYYPRNSIDTKEYMSTLTYGFNGNV
TGDDTGKIGGLIGANVSIGHTLKYVQPDFKTILESPTDKKVGWKVIFNNM
VNQNWGPYDRDSWNPVYGNQLFMKTRNGSMKAADNFLDPNKASSLLSSGF
SPDFATVITMDRKASKQQTNIDVIYERVRDDYQLHWTSTNWKGTNTKDKW
IDRSSERYKIDWEKEEMTN

Hla is an important virulence determinant produced by most strains of S. aureus, having pore-forming and haemolytic activity. Anti-Hla antibodies can neutralise the detrimental effects of the toxin in animal models, and Hla is particularly useful for protecting against pneumonia.

Hla's natural toxicity can be avoided in compositions of the invention by chemical inactivation (e.g. using formaldehyde, glutaraldehyde or other cross-linking reagents), but it is preferred to use a mutant Hla which lacks Hla's natural toxic activity while retaining its immunogenicity. Such detoxified mutants are already known in the art. A preferred Hla antigen is a mutant S. aureus hemolysin having a mutation at residue 61 of SEQ ID NO: 28, which is residue 35 of the mature antigen (i.e. after omitting the first 26 N-terminal amino acids). Thus residue 61 may not be histidine, and may instead be e.g. Ile, Val or preferably Leu. A His-Arg mutation at this position can also be used. For example, SEQ ID NO: 29 is the mature mutant Hla-H35L sequence:

ADSDINIKTGTTDIGSNTTVKTGDLVTYDKENGMLKKVFYSFIDDKNHNK
KLLVIRTKGTIAGQYRVYSEEGANKSGLAWPSAFKVQLQLPDNEVAQISD
YYPRNSIDTKEYMSTLTYGFNGNVTGDDTGKIGGLIGANVSIGHTLKYVQ
PDFKTILESPTDKKVGWKVIFNNMVNQNWGPYDRDSWNPVYGNQLFMKTR
NGSMKAADNFLDPNKASSLLSSGFSPDFATVITMDRKASKQQTNIDVIYE
RVRDDYQLHWTSTNWKGTNTKDKWIDRSSERYKIDWEKEEMTN

and a useful Hla antigen comprises SEQ ID NO: 29. Other useful mutants are disclosed in reference 1.

Hla mutants used with the invention can elicit an antibody (e.g. when administered to a human) that recognises SEQ ID NO: 28 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 28; and/or (b) comprising a fragment of at least ‘n’ consecutive amino acids of SEQ ID NO: 28, wherein ‘n’ is 7 or more (e.g. 8, 10, 12, 14, 16, 18, 20, 25, 30, 35, 40, 50, 60, 70, 80, 90, 100, 150, 200, 250 or more). These Hla antigens include variants of SEQ ID NO: 28. Preferred fragments of (b) comprise an epitope from SEQ ID NO: 28. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 28 while retaining at least one epitope of SEQ ID NO: 28. The first 26 N-terminal amino acids of SEQ ID NO: 28 can usefully be omitted. Truncation at the C-terminus can also be used e.g. leaving only 50 amino acids (residues 27-76 of SEQ ID NO: 28) [53]. Further useful Hla antigens are disclosed in references 54 and 55.

One useful Hla sequence is SEQ ID NO: 30:

MASADSDINIKTGTTDIGSNTTVKTGDLVTYDKENGMLKKVFYSFIDDKN
HNKKLLVIRTKGTIAGQYRVYSEEGANKSGLAWPSAFKVQLQLPDNEVAQ
ISDYYPRNSIDTKEYMSTLTYGFNGNVTGDDTGKIGGLIGANVSIGHTLK
YVQPDFKTILESPTDKKVGWKVIFNNMVNQNWGPYDRDSWNPVYGNQLFM
KTRNGSMKAADNFLDPNKASSLLSSGFSPDFATVITMDRKASKQQTNIDV
IYERVRDDYQLHWTSTNWKGTNTKDKWIDRSSERYKIDWEKEEMTN

This has a N-terminal Met, then an Ala-Ser dipeptide from the expression vector, then SEQ ID NO: 29.

The ‘Sta006’ antigen is annotated as ‘ferrichrome-binding protein’, and has also been referred to as ‘FhuD2’ [56]. In the NCTC 8325 strain Sta006 has amino acid sequence SEQ ID NO: 31 (GI:88196199):

MKKLLLPLIIMLLVLAACGNQGEKNNKAETKSYKMDDGKTVDIPKDPKRI
AVVAPTYAGGLKKLGANIVAVNQQVDQSKVLKDKFKGVTKIGDGDVEKVA
KEKPDLIIVYSTDKDIKKYQKVAPTVVVDYNKHKYLEQQEMLGKIVGKED
KVKAWKKDWEETTAKDGKEIKKAIGQDATVSLFDEFDKKLYTYGDNWGRG
GEVLYQAFGLKMQPEQQKLTAKAGWAEVKQEEIEKYAGDYIVSTSEGKPT
PGYESTNMWKNLKATKEGHIVKVDAGTYWYNDPYTLDFMRKDLKEKLIKA
AK

Sta006 used with the present invention can elicit an antibody (e.g. when administered to a human) that recognises SEQ ID NO: 31 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 31; and/or (b) comprising a fragment of at least ‘n’ consecutive amino acids of SEQ ID NO: 31, wherein ‘n’ is 7 or more (e.g. 8, 10, 12, 14, 16, 18, 20, 25, 30, 35, 40, 50, 60, 70, 80, 90, 100, 150, 200, 250 or more). These Sta006 polypeptides include variants of SEQ ID NO: 31. Preferred fragments of (b) comprise an epitope from SEQ ID NO: 31. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 31 while retaining at least one epitope of SEQ ID NO: 31. The first 17 N-terminal amino acids of SEQ ID NO: 31 can usefully be omitted. Mutant forms of Sta006 are reported in reference 57. One useful Sta006 sequence is SEQ ID NO: 32, which has a Met-Ala-Ser- sequence at the N-terminus and omits the N-terminus of SEQ ID NO: 31:

MASCGNQGEKNNKAETKSYKMDDGKTVDIPKDPKRIAVVAPTYAGGLKKL
GANIVAVNQQVDQSKVLKDKFKGVTKIGDGDVEKVAKEKPDLIIVYSTDK
DIKKYQKVAPTVVVDYNKHKYLEQQEMLGKIVGKEDKVKAWKKDWEETTA
KDGKEIKKAIGQDATVSLFDEFDKKLYTYGDNWGRGGEVLYQAFGLKMQP
EQQKLTAKAGWAEVKQEEIEKYAGDYIVSTSEGKPTPGYESTNMWKNLKA
TKEGHIVKVDAGTYWYNDPYTLDFMRKDLKEKLIKAAK

SEQ ID NO: 33 is another such sequence, but it lacks the cysteine present in SEQ ID NO: 32:

MASGNQGEKNNKAETKSYKMDDGKTVDIPKDPKRIAVVAPTYAGGLKKLG
ANIVAVNQQVDQSKVLKDKFKGVTKIGDGDVEKVAKEKPDLIIVYSTDKD
IKKYQKVAPTVVVDYNKHKYLEQQEMLGKIVGKEDKVKAWKKDWEETTAK
DGKEIKKAIGQDATVSLFDEFDKKLYTYGDNWGRGGEVLYQAFGLKMQPE
QQKLTAKAGWAEVKQEEIEKYAGDYIVSTSEGKPTPGYESTNMWKNLKAT
KEGHIVKVDAGTYWYNDPYTLDFMRKDLKEKLIKAAK

The ‘Sta011’ antigen has amino acid sequence SEQ ID NO: 34 (GI:88193872) in NCTC 8325:

MMKRLNKLVLGIIFLFLVISITAGCGIGKEAEVKKSFEKTLSMYPIKNLE
DLYDKEGYRDDQFDKNDKGTWIINSEMVIQPNNEDMVAKGMVLYMNRNTK
TTNGYYYVDVTKDEDEGKPHDNEKRYPVKMVDNKIIPTKEIKDEKIKKEI
ENFKFFVQYGDFKNLKNYKDGDISYNPEVPSYSAKYQLTNDDYNVKQLRK
RYDIPTSKAPKLLLKGSGNLKGSSVGYKDIEFTFVEKKEENIYFSDSLDY
KKSGDV

Sta011 antigens used with the invention can elicit an antibody (e.g. when administered to a human) that recognises SEQ ID NO: 34 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 34; and/or (b) comprising a fragment of at least ‘n’ consecutive amino acids of SEQ ID NO: 34, wherein ‘n’ is 7 or more (e.g. 8, 10, 12, 14, 16, 18, 20, 25, 30, 35, 40, 50, 60, 70, 80, 90, 100, 150, 200, 250 or more). These Sta011 polypeptides include variants of SEQ ID NO: 34. Preferred fragments of (b) comprise an epitope from SEQ ID NO: 34. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 34 while retaining at least one epitope of SEQ ID NO: 34. The first 23 N-terminal amino acids of SEQ ID NO: 34 can usefully be omitted. One useful Sta011 sequence is SEQ ID NO: 35, which has a N-terminus methionine and omits the N-terminus of SEQ ID NO: 34:

MGCGIGKEAEVKKSFEKTLSMYPIKNLEDLYDKEGYRDDQFDKNDKGTWI
INSEMVIQPNNEDMVAKGMVLYMNRNTKTTNGYYYVDVTKDEDEGKPHDN
EKRYPVKMVDNKIIPTKEIKDEKIKKEIENFKFFVQYGDFKNLKNYKDGD
ISYNPEVPSYSAKYQLTNDDYNVKQLRKRYDIPTSKAPKLLLKGSGNLKG
SSVGYKDIEFTFVEKKEENIYFSDSLDYKKSGDV

SEQ ID NO: 36 is another such sequence, but it lacks the cysteine present in SEQ ID NO: 35:

MGSGIGKEAEVKKSFEKTLSMYPIKNLEDLYDKEGYRDDQFDKNDKGTWI
INSEMVIQPNNEDMVAKGMVLYMNRNTKTTNGYYYVDVTKDEDEGKPHDN
EKRYPVKMVDNKIIPTKEIKDEKIKKEIENFKFFVQYGDFKNLKNYKDGD
ISYNPEVPSYSAKYQLTNDDYNVKQLRKRYDIPTSKAPKLLLKGSGNLKG
SSVGYKDIEFTFVEKKEENIYFSDSLDYKKSGDV

Sta011 can exist as a monomer or an oligomer, with Ca⁺⁺ ions favouring oligomerisation. The invention can use monomers and/or oligomers of Sta011.

The ‘EsxA’ antigen in the NCTC 8325 strain has amino acid sequence SEQ ID NO: 37 (GI:88194063):

MAMIKMSPEEIRAKSQSYGQGSDQIRQILSDLTRAQGEIAANWEGQAFSR
FEEQFQQLSPKVEKFAQLLEEIKQQLNSTADAVQEQDQQLSNNFGLQ

EsxA antigens used with the present invention can elicit an antibody (e.g. when administered to a human) that recognises SEQ ID NO: 37 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 37; and/or (b) comprising a fragment of at least ‘n’ consecutive amino acids of SEQ ID NO: 37, wherein ‘n’ is 7 or more (e.g. 8, 10, 12, 14, 16, 18, 20, 25, 30, 35, 40, 50, 60, 70, 80, 90 or more). These EsxA polypeptides include variants of SEQ ID NO: 37. Preferred fragments of (b) comprise an epitope from SEQ ID NO: 37. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 37 while retaining at least one epitope of SEQ ID NO: 37.

The ‘EsxB’ antigen in the NCTC 8325 strain has amino acid sequence SEQ ID NO: 38 (GI:88194070):

MGGYKGIKADGGKVDQAKQLAAKTAKDIEACQKQTQQLAEYIEGSDWEGQ
FANKVKDVLLIMAKFQEELVQPMADHQKAIDNLSQNLAKYDTLSIKQGLD
RVNP

EsxB used with the present invention can elicit an antibody (e.g. when administered to a human) that recognises SEQ ID NO: 38 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 38; and/or (b) comprising a fragment of at least ‘n’ consecutive amino acids of SEQ ID NO: 38, wherein ‘n’ is 7 or more (e.g. 8, 10, 12, 14, 16, 18, 20, 25, 30, 35, 40, 50, 60, 70, 80, 90, 100 or more). These EsxB polypeptides include variants of SEQ ID NO: 38. Preferred fragments of (b) comprise an epitope from SEQ ID NO: 38. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 38 while retaining at least one epitope of SEQ ID NO: 38.

Where a composition includes both EsxA and EsxB antigens, these may be present as a single polypeptide (i.e. as a fusion polypeptide). Thus a single polypeptide can elicit antibodies (e.g. when administered to a human) that recognise both SEQ ID NO: 37 and SEQ ID NO: 38. The single polypeptide can include: (i) a first polypeptide sequence having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 37 and/or comprising a fragment of at least ‘n’ consecutive amino acids of SEQ ID NO: 37, as defined above for EsxA; and (ii) a second polypeptide sequence having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 38 and/or comprising a fragment of at least ‘n’ consecutive amino acids of SEQ ID NO: 38, as defined above for EsxB. The first and second polypeptide sequences can be in either order, N- to C-terminus. SEQ ID NO: 39 (‘EsxAB’) is an example of such a polypeptides, having hexapeptide linkers ASGGGS (SEQ ID NO: 40):

MAMIKMSPEEIRAKSQSYGQGSDQIRQILSDLTRAQGEIAANWEGQAFSR
FEEQFQQLSPKVEKFAQLLEEIKQQLNSTADAVQEQDQQLSNNFGLQASG
GGSMGGYKGIKADGGKVDQAKQLAAKTAKDIEACQKQTQQLAEYIEGSDW
EGQFANKVKDVLLIMAKFQEELVQPMADHQKAIDNLSQNLAKYDTLSIKQ
GLDRVNP

Another ‘EsxAB’ hybrid comprises SEQ ID NO: 41:

AMIKMSPEEIRAKSQSYGQGSDQIRQILSDLTRAQGEIAANWEGQAFSRF
EEQFQQLSPKVEKFAQLLEEIKQQLNSTADAVQEQDQQLSNNFGLQASGG
GSGGYKGIKADGGKVDQAKQLAAKTAKDIEACQKQTQQLAEYIEGSDWEG
QFANKVKDVLLIMAKFQEELVQPMADHQKAIDNLSQNLAKYDTLSIKQGL
DRVNP

which may additionally be provided with a N-terminus methionine (SEQ ID NO: 42):

MAMIKMSPEEIRAKSQSYGQGSDQIRQILSDLTRAQGEIAANWEGQAFSR
FEEQFQQLSPKVEKFAQLLEEIKQQLNSTADAVQEQDQQLSNNFGLQASG
GGSGGYKGIKADGGKVDQAKQLAAKTAKDIEACQKQTQQLAEYIEGSDWE
GQFANKVKDVLLIMAKFQEELVQPMADHQKAIDNLSQNLAKYDTLSIKQG
LDRVNP

A useful variant of EsxAB lacks the internal cysteine residue of EsxB e.g. SEQ ID NO: 43:

MAMIKMSPEEIRAKSQSYGQGSDQIRQILSDLTRAQGEIAANWEGQAFSR
FEEQFQQLSPKVEKFAQLLEEIKQQLNSTADAVQEQDQQLSNNFGLQASG
GGSGGYKGIKADGGKVDQAKQLAAKTAKDIEAAQKQTQQLAEYIEGSDWE
GQFANKVKDVLLIMAKFQEELVQPMADHQKAIDNLSQNLAKYDTLSIKQG
LDRVNP

Thus a useful polypeptide comprises an amino acid sequence (a) having 80% or more identity (e.g. 80%, 85%, 90%, 91%, 92%, 93%, 94%, 95%, 96%, 97%, 98%, 99%, 99.5% or more) to SEQ ID NO: 41; and/or (b) comprising both a fragment of at least ‘n’ consecutive amino acids from amino acids 1-96 of SEQ ID NO: 41 and a fragment of at least ‘n’ consecutive amino acids from amino acids 103-205 of SEQ ID NO: 41, wherein ‘n’ is 7 or more (e.g. 8, 10, 12, 14, 16, 18, 20, 25, 30, 35, 40, 50, 60, 70, 80, 90, 100, 150, 200, 250 or more). These polypeptides (e.g. SEQ ID NO: 42) can elicit antibodies (e.g. when administered to a human) which recognise both the wild-type staphylococcal protein comprising SEQ ID NO: 37 and the wild-type staphylococcal protein comprising SEQ ID NO: 38. Thus the immune response will recognise both of antigens EsxA and EsxB. Preferred fragments of (b) provide an epitope from SEQ ID NO: 37 and an epitope from SEQ ID NO: 38.

Although SEQ ID NOs: 30, 32, 35 and 42 are useful amino acid sequences in a combination, the invention is not limited to these precise sequences. Thus 1, 2, 3 or all 4 of these sequences can independently be modified by up to 5 single amino changes (i.e. 1, 2, 3, 4 or 5 single amino acid substitutions, deletions and/or insertions) provided that the modified sequence can elicit antibodies which still bind to a polypeptide consisting of the unmodified sequence. For instance, SEQ ID NOs: 33, 36 and 43 are such variants of SEQ ID NOs: 32, 35 and 42.

In a preferred embodiment, the invention provides an immunogenic composition which comprises: (a) a polypeptide of the invention, comprising a CnaBE3 domain: (b) a mutant hemolysin, comprising SEQ ID NO: 30; (c) a sta006 antigen, comprising SEQ ID NO: 32; (d) a sta011 antigen, comprising SEQ ID NO: 35; and (d) an EsxAB antigen, comprising SEQ ID NO: 42.

In another preferred embodiment, the invention provides an immunogenic composition which comprises: (a) a polypeptide of the invention, comprising a CnaBE3 domain: (b) a mutant hemolysin, comprising SEQ ID NO: 30; (c) a sta006 antigen, comprising SEQ ID NO: 33; (d) a sta011 antigen, comprising SEQ ID NO: 36; and (d) an EsxAB antigen, comprising SEQ ID NO: 43.

Combinations with Non-Staphylococcal Antigens

The individual antigens identified in the antigen groups of the invention may be used in combination with non-staphylococcal antigens, and in particular with antigens from bacteria associated with nosocomial infections. Thus the invention provides an immunogenic composition comprising a combination of:
(1) a polypeptide of the invention; and
(2) one or more antigen(s) selected from the group consisting of: Clostridium difficile; Pseudomonas aeruginosa; Candida albicans; and extraintestinal pathogenic Escherichia coli.

Further suitable antigens for use in combination with staphylococcal antigens of the invention are listed on pages 33-46 of reference 58.

Polypeptides Used with the Invention

Polypeptides used with the invention can take various forms (e.g. native, fusions, glycosylated, non-glycosylated, lipidated, non-lipidated, phosphorylated, non-phosphorylated, myristoylated, non-myristoylated, monomeric, multimeric, particulate, denatured, etc.).

Polypeptides used with the invention can be prepared by various means (e.g. recombinant expression, purification from cell culture, chemical synthesis, etc.). Recombinantly-expressed proteins are preferred.

Polypeptides used with the invention are preferably provided in purified or substantially purified form i.e. substantially free from other polypeptides (e.g. free from naturally-occurring polypeptides), particularly from other staphylococcal or host cell polypeptides, and are generally at least about 50% pure (by weight), and usually at least about 90% pure i.e. less than about 50%, and more preferably less than about 10% (e.g. 5%) of a composition is made up of other expressed polypeptides. Thus the antigens in the compositions are separated from the whole organism with which the molecule is expressed.

Polypeptides used with the invention are preferably staphylococcal polypeptides.

The term “polypeptide” refers to amino acid polymers of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included are, for example, polypeptides containing one or more analogs of an amino acid (including, for example, unnatural amino acids, etc.), as well as other modifications known in the art. Polypeptides can occur as single chains or associated chains.

The invention provides polypeptides comprising a sequence -P-Q- or -Q-P-, wherein: -P- is an amino acid sequence as defined above and -Q- is not a sequence as defined above i.e. the invention provides fusion proteins. Where the N-terminus codon of -P- is not ATG, but this codon is not present at the N-terminus of a polypeptide, it will be translated as the standard amino acid for that codon rather than as a Met. Where this codon is at the N-terminus of a polypeptide, however, it will be translated as Met. Examples of -Q- moieties include, but are not limited to, histidine tags (i.e. His (SEQ ID NO: 45) where n=3, 4, 5, 6, 7, 8, 9, 10 or more), maltose-binding protein, or glutathione-S-transferase (GST).

The invention also provides a process for producing a polypeptide of the invention, comprising the step of culturing a host cell transformed with nucleic acid of the invention under conditions which induce polypeptide expression.

Although expression of the polypeptides of the invention can take place in a Staphylococcus, the invention will usually use a heterologous host for expression (recombinant expression). The heterologous host may be prokaryotic (e.g. a bacterium) or eukaryotic. It may be E. coli, but other suitable hosts include Bacillus subtilis, Vibrio cholerae, Salmonella typhi, Salmonella typhimurium, Neisseria lactamica, Neisseria cinerea, Mycobacteria (e.g. M. tuberculosis), yeasts, etc. Compared to the wild-type S. aureus genes encoding polypeptides of the invention, it is helpful to change codons to optimise expression efficiency in such hosts without affecting the encoded amino acids.

The invention provides a process for producing a polypeptide of the invention, comprising the step of synthesising at least part of the polypeptide by chemical means.

### Nucleic Acids

The invention also provides nucleic acid encoding polypeptides of the invention. It also provides nucleic acid comprising a nucleotide sequence that encodes one or more polypeptides of the invention.

Nucleic acids of the invention are preferably provided in purified or substantially purified form i.e. substantially free from other nucleic acids (e.g. free from naturally-occurring nucleic acids), particularly from other staphylococcal or host cell nucleic acids, generally being at least about 50% pure (by weight), and usually at least about 90% pure. Nucleic acids of the invention are preferably staphylococcal nucleic acids.

Nucleic acids of the invention may be prepared in many ways e.g. by chemical synthesis (e.g. phosphoramidite synthesis of DNA) in whole or in part, by digesting longer nucleic acids using nucleases (e.g. restriction enzymes), by joining shorter nucleic acids or nucleotides (e.g. using ligases or polymerases), from genomic or cDNA libraries, etc.

The term “nucleic acid” includes in general means a polymeric form of nucleotides of any length, which contain deoxyribonucleotides, ribonucleotides, and/or their analogs. It includes DNA, RNA, DNA/RNA hybrids. It also includes DNA or RNA analogs, such as those containing modified backbones (e.g. peptide nucleic acids (PNAs) or phosphorothioates) or modified bases. Thus the invention includes mRNA, tRNA, rRNA, ribozymes, DNA, cDNA, recombinant nucleic acids, branched nucleic acids, plasmids, vectors, probes, primers, etc. Where nucleic acid of the invention takes the form of RNA, it may or may not have a 5′ cap.

Nucleic acids of the invention may be part of a vector i.e. part of a nucleic acid construct designed for transduction/transfection of one or more cell types. Vectors may be, for example, “cloning vectors” which are designed for isolation, propagation and replication of inserted nucleotides, “expression vectors” which are designed for expression of a nucleotide sequence in a host cell, “viral vectors” which is designed to result in the production of a recombinant virus or virus-like particle, or “shuttle vectors”, which comprise the attributes of more than one type of vector. Preferred vectors are plasmids. A “host cell” includes an individual cell or cell culture which can be or has been a recipient of exogenous nucleic acid. Host cells include progeny of a single host cell, and the progeny may not necessarily be completely identical (in morphology or in total DNA complement) to the original parent cell due to natural, accidental, or deliberate mutation and/or change. Host cells include cells transfected or infected in vivo or in vitro with nucleic acid of the invention.

Nucleic acids of the invention can be used, for example: to produce polypeptides; as hybridization probes for the detection of nucleic acid in biological samples; to generate additional copies of the nucleic acids; to generate ribozymes or antisense oligonucleotides; as single-stranded DNA primers or probes; or as triple-strand forming oligonucleotides.

The invention provides a process for producing nucleic acid of the invention, wherein the nucleic acid is synthesised in part or in whole using chemical means.

The invention provides vectors comprising nucleotide sequences of the invention (e.g. cloning or expression vectors) and host cells transformed with such vectors.

Nucleic acid amplification according to the invention may be quantitative and/or real-time.

### Strains and Variants

Genome sequences of several strains of S. aureus are available, including those of MRSA strains N315 and Mu50 [59], MW2, N315, COL, MRSA252, MSSA476, RF122, USA300 (very virulent), JH1 and JH9. Standard search and alignment techniques can be used to identify in any of these (or other) further genome sequences the homolog of SdrE (SEQ ID NO: 1) from the Newman strain. Moreover, the available sequences from the Newman strain can be used to design primers for amplification of homologous sequences from other strains. Thus the invention is not limited to this strain, but rather encompasses such variants and homologs from other strains of S. aureus, as well as non-natural variants. In general, suitable variants of SEQ ID NO: 1 include its allelic variants, its polymorphic forms, its homologs, its orthologs, its paralogs, its mutants, etc.

Thus, for instance, polypeptides used with the invention may, compared to the SEQ ID NOs herein, include one or more (e.g. 1, 2, 3, 4, 5, 6, 7, 8, 9, etc.) amino acid substitutions, such as conservative substitutions (i.e. substitutions of one amino acid with another which has a related side chain). Genetically-encoded amino acids are generally divided into four families: (1) acidic i.e. aspartate, glutamate; (2) basic i.e. lysine, arginine, histidine; (3) non-polar i.e. alanine, valine, leucine, isoleucine, proline, phenylalanine, methionine, tryptophan; and (4) uncharged polar i.e. glycine, asparagine, glutamine, cysteine, serine, threonine, tyrosine. Phenylalanine, tryptophan, and tyrosine are sometimes classified jointly as aromatic amino acids. In general, substitution of single amino acids within these families does not have a major effect on the biological activity. The polypeptides may also include one or more (e.g. 1, 2, 3, 4, 5, 6, 7, 8, 9, etc.) single amino acid deletions relative to the SEQ ID NO sequences. The polypeptides may also include one or more (e.g. 1, 2, 3, 4, 5, 6, 7, 8, 9, etc.) insertions (e.g. each of 1, 2, 3, 4 or 5 amino acids) relative to the SEQ ID NO sequences.

Similarly, a polypeptide used with the invention may comprise an amino acid sequence that:
is identical (i.e. 100% identical) to a sequence disclosed in the sequence listing;
shares sequence identity (e.g. 80%, 85%, 90%, 91%, 92%, 93%, 94%, 95%, 96%, 97%, 98%, 99%, 99.5% or more) with a sequence disclosed in the sequence listing;
has 1, 2, 3, 4, 5, 6, 7, 8, 9 or 10 (or more) single amino acid alterations (deletions, insertions, substitutions), which may be at separate locations or may be contiguous, as compared to the sequences of (a) or (b); and
when aligned with a particular sequence from the sequence listing using a pairwise alignment algorithm, each moving window of x amino acids from N-terminus to C-terminus (such that for an alignment that extends top amino acids, where p>x, there are p-x+1 such windows) has at least xy identical aligned amino acids, where: x is selected from 20, 25, 30, 35, 40, 45, 50, 60, 70, 80, 90, 100, 150, 200; y is selected from 0.50, 0.60, 0.70, 0.75, 0.80, 0.85, 0.90, 0.91, 0.92, 0.93, 0.94, 0.95, 0.96, 0.97, 0.98, 0.99; and if xy is not an integer then it is rounded up to the nearest integer. The preferred pairwise alignment algorithm is the Needleman-Wunsch global alignment algorithm [60], using default parameters (e.g. with Gap opening penalty=10.0, and with Gap extension penalty=0.5, using the EBLOSUM62 scoring matrix). This algorithm is conveniently implemented in the needle tool in the EMBOSS package [61].

Within group (c), deletions or substitutions may be at the N-terminus and/or C-terminus, or may be between the two termini. Thus a truncation is an example of a deletion. Truncations may involve deletion of up to 40 (or more) amino acids at the N-terminus and/or C-terminus. N-terminus truncation can remove leader peptides e.g. to facilitate recombinant expression in a heterologous host. C-terminus truncation can remove anchor sequences e.g. to facilitate recombinant expression in a heterologous host.

In general, when an antigen comprises a sequence that is not identical to a complete S. aureus sequence from the sequence listing (e.g. when it comprises a sequence listing with <100% sequence identity thereto, or when it comprises a fragment thereof) it is preferred in each individual instance that the antigen can elicit an antibody which recognises the respective complete S. aureus sequence.

### Immunogenic Compositions and Medicaments

Polypeptides of the invention are useful as components in immunogenic compositions. Immunogenic compositions of the invention may be useful as vaccines. Vaccines according to the invention may either be prophylactic (i.e. to prevent infection) or therapeutic (i.e. to treat infection), but will typically be prophylactic.

Compositions may thus be pharmaceutically acceptable. They will usually include components in addition to the antigens e.g. they typically include one or more pharmaceutical carrier(s) and/or excipient(s). A thorough discussion of such components is available in reference 62.

Compositions will generally be in aqueous form, particularly at the point of administration, but they can also be presented in non-aqueous liquid forms or in dried forms e.g. as lyophilisates. Some vaccines are manufactured in aqueous form, then filled and distributed and administered also in aqueous form, but other vaccines are lyophilised during manufacture and are reconstituted into an aqueous form at the time of use. Thus a composition of the invention may be dried, such as a lyophilised formulation.

The composition may include preservatives such as thiomersal or 2-phenoxyethanol. It is preferred, however, that the vaccine should be substantially free from (i.e. less than 5 μg/ml) mercurial material e.g. thiomersal-free. Vaccines containing no mercury are more preferred. Preservative-free vaccines are particularly preferred.

To improve thermal stability, a composition may include a temperature protective agent.

To control tonicity, it is preferred to include a physiological salt, such as a sodium salt e.g. to control tonicity. Sodium chloride (NaCl) is preferred, which may be present at between 1 and 20 mg/ml e.g. about 10±2 mg/ml NaCl, or 9 mg/ml. Other salts that may be present include potassium chloride, potassium dihydrogen phosphate, disodium phosphate dehydrate, magnesium chloride, calcium chloride, etc.

Compositions will generally have an osmolality of between 200 mOsm/kg and 400 mOsm/kg, preferably between 240-360 mOsm/kg, or between 290-310 mOsm/kg.

Compositions may include polypeptides in plain water (e.g. w.f.i.) but will usually include one or more buffers. Typical buffers include: a phosphate buffer; a Tris buffer; a borate buffer; a succinate buffer; a histidine buffer (particularly with an aluminum hydroxide adjuvant); or a citrate buffer. Buffers will typically be included in the 5-20 mM range.

The pH of a composition will generally be between 5.0 and 8.1, and more typically between 6.0 and 8.0 e.g. 6.5 and 7.5, or between 7.0 and 7.8.

The composition is preferably sterile. The composition is preferably non-pyrogenic e.g. containing <1 EU (endotoxin unit, a standard measure) per dose, and preferably <0.1 EU per dose. The composition is preferably gluten free.

Compositions should be suitable for administration to animal (and, in particular, human) patients, and thus include both human and veterinary uses. They may be used in a method of raising an immune response in a patient, comprising the step of administering the composition to the patient (see below). Compositions may be administered before a subject is exposed to a pathogen and/or after a subject is exposed to a pathogen.

Pharmaceutical compositions may be prepared in unit dose form. In some embodiments a unit dose may have a volume of between 0.1-1.0 ml e.g. about 0.5 ml.

The composition may include material for a single immunisation, or may include material for multiple immunisations (i.e. a ‘multidose’ kit). The inclusion of a preservative is preferred in multidose arrangements. As an alternative (or in addition) to including a preservative in multidose compositions, the compositions may be contained in a container having an aseptic adaptor for removal of material.

Human vaccines are typically administered in a dosage volume of about 0.5 ml, although a half dose (i.e. about 0.25 ml) may be administered to children.

The invention also provides a delivery device (e.g. syringe, nebuliser, sprayer, inhaler, dermal patch, etc.) containing an immunogenic composition of the invention e.g. containing a unit dose. This device can be used to administer the composition to a mammal.

The invention also provides a sterile container (e.g. a vial) containing an immunogenic composition of the invention e.g. containing a unit dose.

The invention also provides a unit dose of an immunogenic composition of the invention.

The invention also provides a hermetically sealed container containing an immunogenic composition of the invention. Suitable containers include e.g. a vial.

S. aureus infections can affect various areas of the body and so the compositions of the invention may be prepared in various forms. For example, the compositions may be prepared as injectables, either as liquid solutions or suspensions. Solid forms suitable for solution in, or suspension in, liquid vehicles prior to injection can also be prepared (e.g. a lyophilised composition or a spray-freeze dried composition). The composition may be prepared for topical administration. The composition may be prepared for oral administration. The composition may be prepared for nasal administration e.g. as a spray. The composition may be in kit form, designed such that a combined composition is reconstituted just prior to administration to a patient. Such kits may comprise one or more antigens in liquid form and one or more lyophilised antigens.

Where a composition is to be prepared extemporaneously prior to use (e.g. where a component is presented in lyophilised form) and is presented as a kit, the kit may comprise two vials, or it may comprise one ready-filled syringe and one vial, with the contents of the syringe being used to reactivate the contents of the vial prior to injection.

Immunogenic compositions used as vaccines comprise an immunologically effective amount of antigen(s), as well as any other components, as needed. By ‘immunologically effective amount’, it is meant that the administration of that amount to an individual, either in a single dose or as part of a series, is effective for treatment or prevention. This amount varies depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the treating doctor's assessment of the medical situation, and other relevant factors. It is expected that the amount will fall in a relatively broad range that can be determined through routine trials. Where more than one antigen is included in a composition then two antigens may be present at the same dose as each other or at different doses.

Immunogenic compositions of the invention will typically include one or more immunological adjuvants. Adjuvants which may be used in compositions of the invention include, but are not limited to: (i) an oil-in-water emulsion (ii) at least one aluminium salt or (iii) at least one TLR agonist. In some embodiments a composition includes a mixture of an aluminium salt and a TLR agonist, and the TLR agonist can be adsorbed to the aluminium salt to improve adjuvant effects [86]. This can lead to a better (stronger, or more quickly achieved) immune response and/or can permit a reduction in the amount of aluminium in the composition while maintaining an equivalent adjuvant effect.

Where a composition includes aluminium salt adjuvant(s) then a polypeptide of the invention can be adsorbed to the salt(s). Where a composition includes an aluminium salt adjuvant then preferably it does not also include an oil-in-water emulsion adjuvant. Conversely, where a composition includes an oil-in-water emulsion adjuvant then preferably it does not also include an aluminium salt adjuvant.

### Oil-in-Water Emulsion Adjuvants

An immunogenic composition can be adjuvanted with an oil-in-water emulsion. Various such emulsions are known e.g. MF59 and AS03 are both authorised in Europe.

Useful emulsion adjuvants they typically include at least one oil and at least one surfactant, with the oil(s) and surfactant(s) being biodegradable (metabolisable) and biocompatible. The oil droplets in the emulsion generally have a sub-micron diameter, and these small sizes can readily be achieved with a microfluidiser to provide stable emulsions, or by alternative methods e.g. phase inversion. Emulsions in which at least 80% (by number) of droplets have a diameter of less than 220 nm are preferred, as they can be subjected to filter sterilization.

The emulsion can include oil(s) from an animal (such as fish) and/or vegetable source. Sources for vegetable oils include nuts, seeds and grains. Peanut oil, soybean oil, coconut oil, and olive oil, the most commonly available, exemplify the nut oils. Jojoba oil can be used e.g. obtained from the jojoba bean. Seed oils include safflower oil, cottonseed oil, sunflower seed oil, sesame seed oil and the like. In the grain group, corn oil is the most readily available, but the oil of other cereal grains such as wheat, oats, rye, rice, teff, triticale and the like may also be used. 6-10 carbon fatty acid esters of glycerol and 1,2-propanediol, while not occurring naturally in seed oils, may be prepared by hydrolysis, separation and esterification of the appropriate materials starting from the nut and seed oils. Fats and oils from mammalian milk are metabolisable and may therefore be used with the invention. The procedures for separation, purification, saponification and other means necessary for obtaining pure oils from animal sources are well known in the art.

Most fish contain metabolisable oils which may be readily recovered. For example, cod liver oil, shark liver oils, and whale oil such as spermaceti exemplify several of the fish oils which may be used herein. A number of branched chain oils are synthesized biochemically in 5-carbon isoprene units and are generally referred to as terpenoids. Shark liver oil contains a branched, unsaturated terpenoids known as squalene, 2,6,10,15,19,23-hexamethyl-2,6,10,14,18,22-tetracosahexaene, which is particularly preferred for use with the invention (see below). Squalane, the saturated analog to squalene, is also a useful oil. Fish oils, including squalene and squalane, are readily available from commercial sources or may be obtained by methods known in the art. Other preferred oils are the tocopherols (see below). Mixtures of oils can be used.

Preferred amounts of total oil (% by volume) in an adjuvant emulsion are between 1 and 20% e.g. between 2-10%. A squalene content of 5% by volume is particularly useful.

Surfactants can be classified by their ‘HLB’ (hydrophile/lipophile balance). Preferred surfactants of the invention have a HLB of at least 10 e.g. about 15. The invention can be used with surfactants including, but not limited to: the polyoxyethylene sorbitan esters surfactants (commonly referred to as the Tweens), especially polysorbate 20 or polysorbate 80; copolymers of ethylene oxide (EO), propylene oxide (PO), and/or butylene oxide (BO), sold under the DOWFAX™ tradename, such as linear EO/PO block copolymers; octoxynols, which can vary in the number of repeating ethoxy(oxy-1,2-ethanediyl) groups, with octoxynol-9 (Triton X-100, or t-octylphenoxypolyethoxyethanol) being of particular interest; (octylphenoxy)polyethoxyethanol (IGEPAL CA-630/NP-40); phospholipids such as phosphatidylcholine (lecithin); nonylphenol ethoxylates, such as the Tergitol™ NP series; polyoxyethylene fatty ethers derived from lauryl, cetyl, stearyl and oleyl alcohols (known as Brij surfactants), such as triethyleneglycol monolauryl ether (Brij 30); and sorbitan esters (commonly known as the Spans), such as sorbitan trioleate (Span 85) or sorbitan monolaurate.

Emulsions used with the invention preferably include non-ionic surfactant(s). Preferred surfactants for including in the emulsion are polysorbate 80 (polyoxyethylene sorbitan monooleate; Tween 80), Span 85 (sorbitan trioleate), lecithin or Triton X-100. Mixtures of surfactants can be used e.g. a mixture of polysorbate 80 and sorbitan trioleate. A combination of a polyoxyethylene sorbitan ester such as polysorbate 80 (Tween 80) and an octoxynol such as t-octylphenoxypolyethoxyethanol (Triton X-100) is also useful. Another useful combination comprises laureth 9 plus a polyoxyethylene sorbitan ester and/or an octoxynol. Where a mixture of surfactants is used then the HLB of the mixture is calculated according to their relative weightings (by volume) e.g. the preferred 1:1 mixture by volume of polysorbate 80 and sorbitan trioleate has a HLB of 8.4.

Preferred amounts of total surfactant (% by volume) in an adjuvant emulsion are between 0.1 and 2% e.g. between 0.25-2%. A total content of 1% by volume is particularly useful e.g. 0.5% by volume of polysorbate 80 and 0.5% by volume of sorbitan trioleate.

Useful emulsions can be prepared using known techniques e.g. see references 63-646984

Specific oil-in-water emulsion adjuvants useful with the invention include, but are not limited to:
A submicron emulsion of squalene, polysorbate 80, and sorbitan trioleate. The composition of the emulsion by volume can be about 5% squalene, about 0.5% polysorbate 80 and about 0.5% sorbitan trioleate. In weight terms, these ratios become 4.3% squalene, 0.5% polysorbate 80 and 0.48% sorbitan trioleate. This adjuvant is known as ‘MF59’ [70-72], as described in more detail in Chapter 10 of ref. 83 and chapter 12 of ref. 84. The MF59 emulsion advantageously includes citrate ions e.g. 10 mM sodium citrate buffer.
An emulsion of squalene, a tocopherol, and polysorbate 80. The emulsion may include phosphate buffered saline. These emulsions may have from 2 to 10% squalene, from 2 to 10% tocopherol and from 0.3 to 3% polysorbate 80, and the weight ratio of squalene:tocopherol is preferably ≦1 (e.g. 0.90) as this can provide a more stable emulsion. Squalene and polysorbate 80 may be present volume ratio of about 5:2, or at a weight ratio of about 11:5. Thus the three components (squalene, tocopherol, polysorbate 80) may be present at a weight ratio of 1068:1186:485 or around 55:61:25. This adjuvant is known as ‘AS03’. Another useful emulsion of this type may comprise, per human dose, 0.5-10 mg squalene, 0.5-11 mg tocopherol, and 0.1-4 mg polysorbate 80 [73] e.g. in the ratios discussed above.
An emulsion in which a saponin (e.g. QuilA or QS21) and a sterol (e.g. a cholesterol) are associated as helical micelles [74].
An emulsion having from 0.5-50% of an oil, 0.1-10% of a phospholipid, and 0.05-5% of a non-ionic surfactant. As described in reference 75, preferred phospholipid components are phosphatidylcholine, phosphatidylethanolamine, phosphatidylserine, phosphatidylinositol, phosphatidylglycerol, phosphatidic acid, sphingomyelin and cardiolipin. Submicron droplet sizes are advantageous.
An emulsion comprising squalene, an aqueous solvent, a polyoxyethylene alkyl ether hydrophilic nonionic surfactant (e.g. polyoxyethylene (12) cetostearyl ether) and a hydrophobic nonionic surfactant (e.g. a sorbitan ester or mannide ester, such as sorbitan monoleate or ‘Span 80’). The emulsion is preferably thermoreversible and/or has at least 90% of the oil droplets (by volume) with a size less than 200 nm [76]. The emulsion may also include one or more of: alditol; a cryoprotective agent (e.g. a sugar, such as dodecylmaltoside and/or sucrose); and/or an alkylpolyglycoside. It may also include a TLR4 agonist, such as one whose chemical structure does not include a sugar ring [77]. Such emulsions may be lyophilized. The ‘AF03’ product is one such emulsion.

Preferred oil-in-water emulsions used with the invention comprise squalene and polysorbate 80.

The emulsions may be mixed with antigens during vaccine manufacture, or they may be mixed extemporaneously at the time of delivery. Thus, in some embodiments, the adjuvant and antigens may be kept separately in a packaged or distributed vaccine, ready for final formulation at the time of use. At the time of mixing (whether during bulk manufacture, or at the point of use) the antigen will generally be in an aqueous form, such that the final vaccine is prepared by mixing two liquids. The volume ratio of the two liquids for mixing can vary (e.g. between 5:1 and 1:5) but is generally about 1:1. If emulsion and antigen are stored separately in a kit then the product may be presented as a vial containing emulsion and a vial containing aqueous antigen, for mixing to give adjuvanted liquid vaccine (monodose or multi-dose).

Preferred emulsions of the invention include squalene oil. This is usually prepared from shark oil but alternative sources are known e.g. see references 78 (yeast) and 79 (olive oil). Squalene which contains less than 661 picograms of PCBs per gram of squalene (TEQ) is preferred for use with the invention, as disclosed in reference 80. The emulsions are preferably made from squalene of high purity e.g. prepared by double-distillation as disclosed in reference 81.

Where a composition includes a tocopherol, any of the α, β, γ, δ, ε or ξ tocopherols can be used, but α-tocopherols are preferred. The tocopherol can take several forms e.g. different salts and/or isomers. Salts include organic salts, such as succinate, acetate, nicotinate, etc. D-α-tocopherol and DL-α-tocopherol can both be used. Tocopherols have antioxidant properties that may help to stabilize the emulsions [82]. A preferred α-tocopherol is DL-α-tocopherol, and a preferred salt of this tocopherol is the succinate.

### Aluminium Salt Adjuvants

Compositions of the invention can include an aluminium salt adjuvant. Aluminium salt adjuvants currently in use are typically referred to either as “aluminium hydroxide” or as “aluminium phosphate” adjuvants. These are names of convenience, however, as neither is a precise description of the actual chemical compound which is present (e.g. see chapter 9 of reference 83, and chapter 4 of reference 84). The invention can use any of the “hydroxide” or “phosphate” salts that useful as adjuvants. Aluminium salts which include hydroxide ions are preferred if adsorption of a TLR agonist is desired as these hydroxide ions can readily undergo ligand exchange for adsorption of the TLR agonist. Thus preferred salts for adsorption of TLR agonists are aluminium hydroxide and/or aluminium hydroxyphosphate. These have surface hydroxyl moieties which can readily undergo ligand exchange with phosphorus-containing groups (e.g. phosphates, phosphonates) to provide stable adsorption. An aluminium hydroxide adjuvant is thus most preferred.

The adjuvants known as “aluminium hydroxide” are typically aluminium oxyhydroxide salts, which are usually at least partially crystalline. Aluminium oxyhydroxide, which can be represented by the formula AlO(OH), can be distinguished from other aluminium compounds, such as aluminium hydroxide Al(OH)₃, by infrared (IR) spectroscopy, in particular by the presence of an adsorption band at 1070 cm⁻¹ and a strong shoulder at 3090-3100 cm⁻¹ (chapter 9 of ref. 83). The degree of crystallinity of an aluminium hydroxide adjuvant is reflected by the width of the diffraction band at half height (WHH), with poorly-crystalline particles showing greater line broadening due to smaller crystallite sizes. The surface area increases as WHH increases, and adjuvants with higher WHH values have been seen to have greater capacity for antigen adsorption. A fibrous morphology (e.g. as seen in transmission electron micrographs) is typical for aluminium hydroxide adjuvants e.g. with needle-like particles with diameters about 2 nm. The PZC of aluminium hydroxide adjuvants is typically about 11 i.e. the adjuvant itself has a positive surface charge at physiological pH. Adsorptive capacities of between 1.8-2.6 mg protein per mg Al⁺⁺⁺ at pH 7.4 have been reported for aluminium hydroxide adjuvants.

The adjuvants known as “aluminium phosphate” are typically aluminium hydroxyphosphates, often also containing a small amount of sulfate. They may be obtained by precipitation, and the reaction conditions and concentrations during precipitation influence the degree of substitution of phosphate for hydroxyl in the salt. Hydroxyphosphates generally have a PO₄/Al molar ratio between 0.3 and 0.99. Hydroxyphosphates can be distinguished from strict AlPO₄ by the presence of hydroxyl groups. For example, an IR spectrum band at 3164 cm⁻¹ (e.g. when heated to 200° C.) indicates the presence of structural hydroxyls (chapter 9 of ref. 83).

The PO₄/Al³⁺ molar ratio of an aluminium phosphate adjuvant will generally be between 0.3 and 1.2, preferably between 0.8 and 1.2, and more preferably 0.95±0.1. The aluminium phosphate will generally be amorphous, particularly for hydroxyphosphate salts. A typical adjuvant is amorphous aluminium hydroxyphosphate with PO₄/Al molar ratio between 0.84 and 0.92, included at 0.6 mg Al³⁺/ml. The aluminium phosphate will generally be particulate. Typical diameters of the particles are in the range 0.5-20 μm (e.g. about 5-10 μm) after any antigen adsorption. Adsorptive capacities of between 0.7-1.5 mg protein per mg Al⁺⁺⁺ at pH 7.4 have been reported for aluminium phosphate adjuvants.

The PZC of aluminium phosphate is inversely related to the degree of substitution of phosphate for hydroxyl, and this degree of substitution can vary depending on reaction conditions and concentration of reactants used for preparing the salt by precipitation. PZC is also altered by changing the concentration of free phosphate ions in solution (more phosphate=more acidic PZC) or by adding a buffer such as a histidine buffer (makes PZC more basic). Aluminium phosphates used according to the invention will generally have a PZC of between 4.0 and 7.0, more preferably between 5.0 and 6.5 e.g. about 5.7.

In solution both aluminium phosphate and hydroxide adjuvants tend to form stable porous aggregates 1-10 μm in diameter [85].

A composition can include a mixture of both an aluminium hydroxide and an aluminium phosphate, and components may be adsorbed to one or both of these salts.

An aluminium phosphate solution used to prepare a composition of the invention may contain a buffer (e.g. a phosphate or a histidine or a Tris buffer), but this is not always necessary. The aluminium phosphate solution is preferably sterile and pyrogen-free. The aluminium phosphate solution may include free aqueous phosphate ions e.g. present at a concentration between 1.0 and 20 mM, preferably between 5 and 15 mM, and more preferably about 10 mM. The aluminium phosphate solution may also comprise sodium chloride. The concentration of sodium chloride is preferably in the range of 0.1 to 100 mg/ml (e.g. 0.5-50 mg/ml, 1-20 mg/ml, 2-10 mg/ml) and is more preferably about 3+1 mg/ml. The presence of NaCl facilitates the correct measurement of pH prior to adsorption of antigens.

A composition of the invention ideally includes less than 0.85 mg Al⁺⁺⁺ per unit dose. In some embodiments of the invention a composition includes less than 0.5 mg Al⁺⁺⁺ per unit dose. The amount of Al⁺⁺⁺ can be lower than this e.g. <250 μg, <200 μg, <150 μg, <100 μg, <75 μg, <50 μg, <25 μg, <10 μg, etc.

Where compositions of the invention include an aluminium-based adjuvant, settling of components may occur during storage. The composition should therefore be shaken prior to administration to a patient. The shaken composition will be a turbid white suspension.

### TLR Agonists

In some embodiments a composition of the invention includes a TLR agonist i.e. a compound which can agonise a Toll-like receptor. Most preferably, a TLR agonist is an agonist of a human TLR. The TLR agonist can activate any of TLR1, TLR2, TLR3, TLR4, TLR5, TLR6, TLR7, TLR8, TLR9 or TLR11; preferably it can activate human TLR4 or human TLR7.

In preferred embodiments, a composition of the invention includes a TLR agonist (such as a TLR7 agonist) which includes a phosphonate group. This phosphonate group can allow adsorption of the agonist to an insoluble aluminium salt [86].

### Methods of Treatment, and Administration of the Vaccine

The invention also provides a method for raising an immune response in a mammal comprising the step of administering an effective amount of an immunogenic composition of the invention. The immune response is preferably protective and preferably involves antibodies and/or cell-mediated immunity. The method may raise a booster response.

The invention also provides an immunogenic composition of the invention for use in therapy e.g. for use in a method for raising an immune response in a mammal (as described above).

The invention also provides the use of a polypeptide of the invention in the manufacture of a medicament for raising an immune response in a mammal (as described above).

By raising an immune response in the mammal by these uses and methods, the mammal can be protected against S. aureus infection, including a nosocomial infection. More particularly, the mammal may be protected against a skin infection, pneumonia, meningitis, osteomyelitis endocarditis, toxic shock syndrome, and/or septicaemia.

The invention also provides a kit comprising a first component and a second component wherein neither the first component nor the second component is a composition of the invention as described above, but wherein the first component and the second component can be combined to provide a composition of the invention as described above. The kit may further include a third component comprising one or more of the following: instructions, syringe or other delivery device, adjuvant, or pharmaceutically acceptable formulating solution.

The mammal is preferably a human. Where the vaccine is for prophylactic use, the human is preferably a child (e.g. a toddler or infant) or a teenager; where the vaccine is for therapeutic use, the human is preferably a teenager or an adult. A vaccine intended for children may also be administered to adults e.g. to assess safety, dosage, immunogenicity, etc. Other mammals which can usefully be immunised according to the invention are cows, dogs, horses, and pigs.

One way of checking efficacy of therapeutic treatment involves monitoring S. aureus infection after administration of the compositions of the invention. One way of checking efficacy of prophylactic treatment involves monitoring immune responses, systemically (such as monitoring the level of IgG1 and IgG2a production) and/or mucosally (such as monitoring the level of IgA production), against the antigens in the compositions of the invention after administration of the composition. Typically, antigen-specific serum antibody responses are determined post-immunisation but pre-challenge whereas antigen-specific mucosal antibody responses are determined post-immunisation and post-challenge.

Another way of assessing the immunogenicity of the compositions of the present invention is to express the proteins recombinantly for screening patient sera or mucosal secretions by immunoblot and/or microarrays. A positive reaction between the protein and the patient sample indicates that the patient has mounted an immune response to the protein in question. This method may also be used to identify immunodominant antigens and/or epitopes within antigens.

The efficacy of vaccine compositions can also be determined in vivo by challenging animal models of S. aureus infection, e.g., guinea pigs or mice, with the vaccine compositions. In particular, there are three useful animal models for the study of S. aureus infectious disease, namely: (i) the murine abscess model [87], (ii) the murine lethal infection model [87] and (iii) the murine pneumonia model [88]. The abscess model looks at abscesses in mouse kidneys after intravenous challenge. The lethal infection model looks at the number of mice which survive after being infected by a normally-lethal dose of S. aureus by the intravenous or intraperitoneal route. The pneumonia model also looks at the survival rate, but uses intranasal infection. A useful vaccine may be effective in one or more of these models. For instance, for some clinical situations it may be desirable to protect against pneumonia, without needing to prevent hematic spread or to promote opsonisation; in other situations the main desire may be to prevent hematic spread. Different antigens, and different antigen combinations, may contribute to different aspects of an effective vaccine.

Compositions of the invention will generally be administered directly to a patient. Direct delivery may be accomplished by parenteral injection (e.g. subcutaneously, intradermally, intraperitoneally, intravenously, intramuscularly, or to the interstitial space of a tissue), or mucosally, such as by rectal, oral (e.g. tablet, spray), vaginal, topical, transdermal or transcutaneous, intranasal, ocular, aural, pulmonary or other mucosal administration. Intramuscular injection is preferred.

The invention may be used to elicit systemic and/or mucosal immunity, preferably to elicit an enhanced systemic and/or mucosal immunity.

Preferably the enhanced systemic and/or mucosal immunity is reflected in an enhanced TH1 and/or TH2 immune response. Preferably, the enhanced immune response includes an increase in the production of IgG1 and/or IgG2a and/or IgA.

Dosage can be by a single dose schedule or a multiple dose schedule. Multiple doses may be used in a primary immunisation schedule and/or in a booster immunisation schedule. In a multiple dose schedule the various doses may be given by the same or different routes e.g. a parenteral prime and mucosal boost, a mucosal prime and parenteral boost, etc. Multiple doses will typically be administered at least 1 week apart (e.g. about 2 weeks, about 3 weeks, about 4 weeks, about 6 weeks, about 8 weeks, about 10 weeks, about 12 weeks, about 16 weeks, etc.).

Vaccines prepared according to the invention may be used to treat both children and adults. Thus a human patient may be less than 1 year old, 1-5 years old, 5-15 years old, 15-55 years old, or at least 55 years old. Preferred patients for receiving the vaccines are the elderly (e.g. ≧50 years old, ≧60 years old, and preferably ≧65 years), the young (e.g. ≧5 years old), hospitalised patients, healthcare workers, armed service and military personnel, pregnant women, the chronically ill, or immunodeficient patients. The vaccines are not suitable solely for these groups, however, and may be used more generally in a population.

Vaccines produced by the invention may be administered to patients at substantially the same time as (e.g. during the same medical consultation or visit to a healthcare professional or vaccination centre) other vaccines e.g. at substantially the same time as an influenza vaccine, a measles vaccine, a mumps vaccine, a rubella vaccine, a MMR vaccine, a varicella vaccine, a MMRV vaccine, a diphtheria vaccine, a tetanus vaccine, a pertussis vaccine, a DTP vaccine, a conjugated H. influenzae type b vaccine, an inactivated poliovirus vaccine, a hepatitis B virus vaccine, a meningococcal conjugate vaccine (such as a tetravalent A-C-W135-Y vaccine), a respiratory syncytial virus vaccine, etc. Further non-staphylococcal vaccines suitable for co-administration may include one or more antigens listed on pages 33-46 of reference 58.

### Nucleic Acid Immunisation

The immunogenic compositions described above include polypeptide antigens from S. aureus. In all cases, however, the polypeptide antigens can be replaced by nucleic acids (typically DNA or RNA) encoding those polypeptides, to give compositions, methods and uses based on nucleic acid immunisation. Nucleic acid immunisation is now a developed field (e.g. see references 89 to 96 etc.).

The nucleic acid encoding the immunogen is expressed in vivo after delivery to a patient and the expressed immunogen then stimulates the immune system. The active ingredient will typically take the form of a nucleic acid vector comprising: (i) a promoter; (ii) a sequence encoding the immunogen, operably linked to the promoter; and optionally (iii) a selectable marker. Preferred vectors may further comprise (iv) an origin of replication; and (v) a transcription terminator downstream of and operably linked to (ii). In general, (i) & (v) will be eukaryotic and (iii) & (iv) will be prokaryotic.

Preferred promoters are viral promoters e.g. from cytomegalovirus (CMV). The vector may also include transcriptional regulatory sequences (e.g. enhancers) in addition to the promoter and which interact functionally with the promoter. Preferred vectors include the immediate-early CMV enhancer/promoter, and more preferred vectors also include CMV intron A. The promoter is operably linked to a downstream sequence encoding an immunogen, such that expression of the immunogen-encoding sequence is under the promoter's control.

Where a marker is used, it preferably functions in a microbial host (e.g. in a prokaryote, in a bacteria, in a yeast). The marker is preferably a prokaryotic selectable marker (e.g. transcribed under the control of a prokaryotic promoter). For convenience, typical markers are antibiotic resistance genes.

The vector of the invention is preferably an autonomously replicating episomal or extrachromosomal vector, such as a plasmid.

The vector of the invention preferably comprises an origin of replication. It is preferred that the origin of replication is active in prokaryotes but not in eukaryotes.

Preferred vectors thus include a prokaryotic marker for selection of the vector, a prokaryotic origin of replication, but a eukaryotic promoter for driving transcription of the immunogen-encoding sequence. The vectors will therefore (a) be amplified and selected in prokaryotic hosts without polypeptide expression, but (b) be expressed in eukaryotic hosts without being amplified. This arrangement is ideal for nucleic acid immunization vectors.

The vector of the invention may comprise a eukaryotic transcriptional terminator sequence downstream of the coding sequence. This can enhance transcription levels. Where the coding sequence does not have its own, the vector of the invention preferably comprises a polyadenylation sequence. A preferred polyadenylation sequence is from bovine growth hormone.

The vector of the invention may comprise a multiple cloning site

In addition to sequences encoding the immunogen and a marker, the vector may comprise a second eukaryotic coding sequence. The vector may also comprise an IRES upstream of said second sequence in order to permit translation of a second eukaryotic polypeptide from the same transcript as the immunogen. Alternatively, the immunogen-coding sequence may be downstream of an IRES.

The vector of the invention may comprise unmethylated CpG motifs e.g. unmethylated DNA sequences which have in common a cytosine preceding a guanosine, flanked by two 5′ purines and two 3′ pyrimidines. In their unmethylated form these DNA motifs have been demonstrated to be potent stimulators of several types of immune cell.

### General

The term “comprising” encompasses “including” as well as “consisting” e.g. a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in relation to a numerical value x is optional and means, for example, x+10%.

Unless specifically stated, a process comprising a step of mixing two or more components does not require any specific order of mixing. Thus components can be mixed in any order. Where there are three components then two components can be combined with each other, and then the combination may be combined with the third component, etc.

Where animal (and particularly bovine) materials are used in the culture of cells, they should be obtained from sources that are free from transmissible spongiform encaphalopathies (TSEs), and in particular free from bovine spongiform encephalopathy (BSE). Overall, it is preferred to culture cells in the total absence of animal-derived materials.

Where a compound is administered to the body as part of a composition then that compound may alternatively be replaced by a suitable prodrug.

In general, the invention will not use a composition which was disclosed in reference 1 or 2. Moreover, in some embodiments the invention does not utilise a CnaB domain which is found within a wild-type SdrC or SdrD protein.

### BRIEF DESCRIPTION OF THE DRAWINGS

FIG. 1 shows log₁₀ CFU/ml in the kidneys of immunised mice after challenge with Newman strain. The three groups of data, from left to right, were immunised with: adjuvant alone; adjuvanted SdrE; or adjuvanted CnaBE3. Each point shows data from a single mouse. The horizontal line is the average.

FIG. 2 shows log₁₀ CFU/ml in the kidneys of immunised mice after challenge with NCTC8325 strain. The two groups of data, from left to right, were immunised with: adjuvant alone; adjuvanted CnaBE3.

FIG. 3 shows a western blot using polyclonal anti-CnaBE3 serum. The table beneath the blot shows the strain being tested, and then the molecular weights of SdrC, SdrD and SdrE in those strains.

FIG. 4 shows % opsonophagocytic killing (Y-axis ranges from -40% to 40%) using indicated sera.

FIG. 5 shows ELISA titres (lnAU) with serum from healthy (left) or infected (right) donors.

### MODES FOR CARRYING OUT THE INVENTION

### SdrE Protein Studies

The coding sequence for SdrE antigen was cloned in a pET15b+ vector in order to encode a protein with a hexahistidine tag (SEQ ID NO: 46) at its N-terminus.

It was noticed that SdrE shows resistance to trypsin digestion. The protein was digested with sequencing-grade modified trypsin (Promega™) overnight at 37° C., using an enzyme/substrate ratio of 1/25 (wt/wt) in 50 mM ammonium bicarbonate, pH 8, with 0.1% (wt/vol) Rapigest (Waters™)

For western blot analysis, bacterial cell-wall extracts were obtained as described previously [97]. S. aureus exponential phase cultures were grown in TSB supplemented with 5 mM CaCl₂ to an OD₆₀₀=0.6. Cells were washed in PBS once and resuspended in 100 μl Lysis Buffer (50 mM Tris-HCl, 20 mM MgCl₂, pH 7.5) supplemented with 30% (w/v) raffinose and 40 μl/ml EDTA-free protease inhibitors cocktail. Lysostaphin (200 μg/ml) was applied for 10 minutes at 37° C. to harvest cell wall proteins. Samples were boiled for 10 min with NuPAGE LDS Sample Buffer and NuPAGE Sample

Reducing Agent and separated in 3-8% (w/v) NuPAGE Tris-Acetate Gels. Electrophoretically separated protein samples were transferred to nitrocellulose membranes with iBlot gel transfer device. Membranes were blocked for 2 hours (25° C., 700 rpm) in 10% (w/v) skim milk in TPBS. After three washes in TPBS, mouse polyclonal anti-rCnaBE3 (diluted 1:1,000 in 1% w/v skim milk) in TPBS was added and membranes were incubated for 1 hour at 25° C., 700 rpm. Membranes were washed three times in TPBS and polyclonal rabbit anti-mouse immunoglobulins-HRP diluted 1:5,000 in 1% (w/v) skim milk in TPBS was added. After 1 hour at 25° C., 700 rpm, membranes were washed three times and bound antibody was visualized through ECL by SuperSignal West Pico Chemiluminescent Substrate and developed for 1 min.

In wild-type bacteria the SdrE protein is visible on western blots as a band around 125 kDa, and it is located in the cell wall fraction. Overnight trypsin treatment provides a strong band at around 36 kDa, with lower weight bands also visible. Even after 3 days of digestion at 37° C., however, the 36 kDa band (and various other bands) remains stable.

MS and N-terminal analysis of the main trypsin-resistant band revealed peptides from the CnaBE3 region, with some sequences extending a short distance into the C-terminal portion of CnaBE2. The BE3 domain was expressed as a 126mer (SEQ ID NO: 27), which includes 15 upstream amino acids from the BE2 domain. The recombinant protein is visible by SDS-PAGE at around 15 kDa. Trypsin digestion reduces its size slightly after 4 hours, but this band remains stable even after 2 days of digestion.

MS studies revealed that the mass of the CnaBE3 peptide differed from the theoretical mass by 17 Da. This mass corresponds to the loss of ammonium which occurs during formation of an isopeptide bond between lysine and asparagine residues. Intramolecular isopeptide bonds in S. aureus proteins have not previously been seen experimentally.

To study possible isopeptide bond formation, six Asn residues within the CnaBE3 domain have been mutated (SEQ ID NOs: 9 to 14). Based on the fact that surface proteins containing CnaA and CnaB domains can form intramolecular isopeptide bonds, and the bonds are formed between Lys-Asp or Lys-Asn residues in presence of Glu/Asp, acting as a stabiliser or catalyst, all the asparagines in the wild-type CnaBE3 were replaced with alanine. The wild-type and mutant CnaBE3 domains (SEQ ID NOs: 9 to 13, where ‘X’ is ‘A’) showed resistance to trypsin digestion, which indicated the presence of some stabilising factor in the CnaBE3 region. Trypsin-resistant behaviour of five mutants suggests that none of these five asparagines is involved in the bond formation.

### Immunological Studies

Full-length SdrE (SEQ ID NO: 1) and the CnaBE3 domain (SEQ ID NO: 27) were adjuvanted with aluminium hydroxide and used to immunise mice. The immunised mice were challenged with the Newman strain and then assessed for kidney abscess formation.

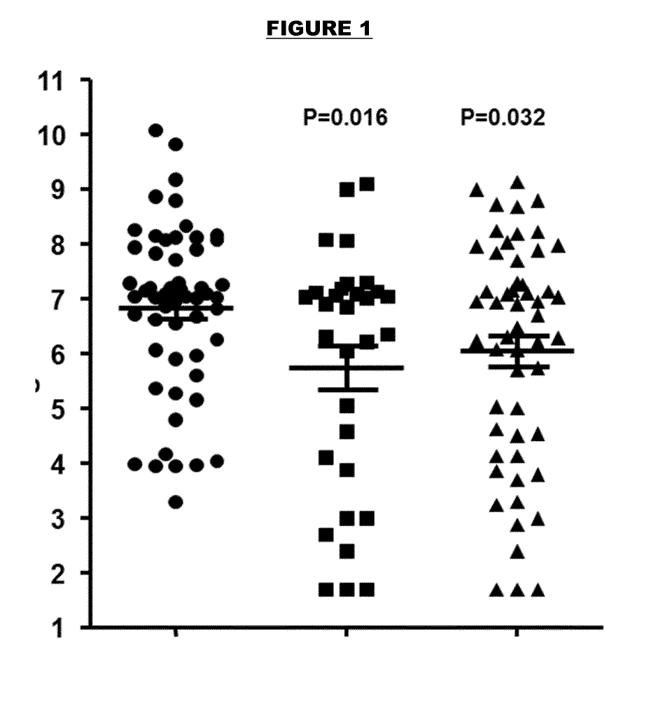

As shown in FIG. 1, immunisation with either SdrE or CnaBE3 led to a significant reduction in bacterial CFU count in kidneys (relative to the negative controls: p=0.016 for SdrE, p=0.032 for CnaBE3). The difference CFU counts in the SdrE and CnaBE3 groups was not significant.

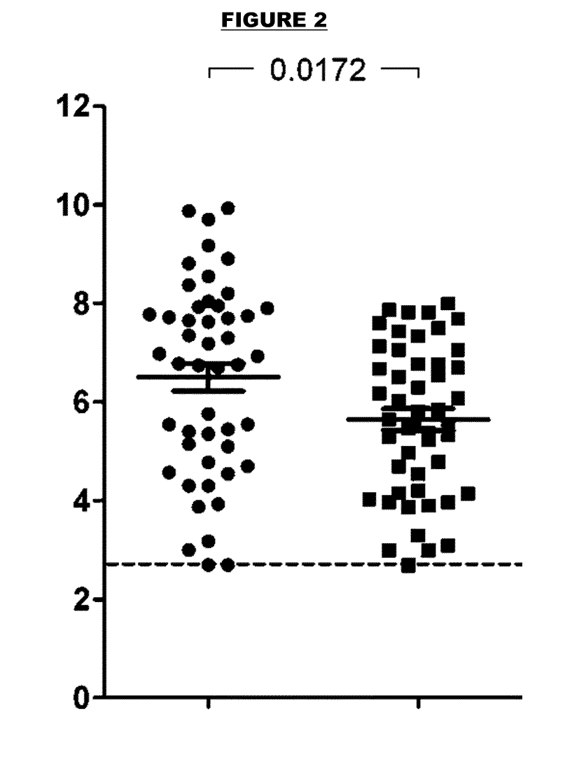

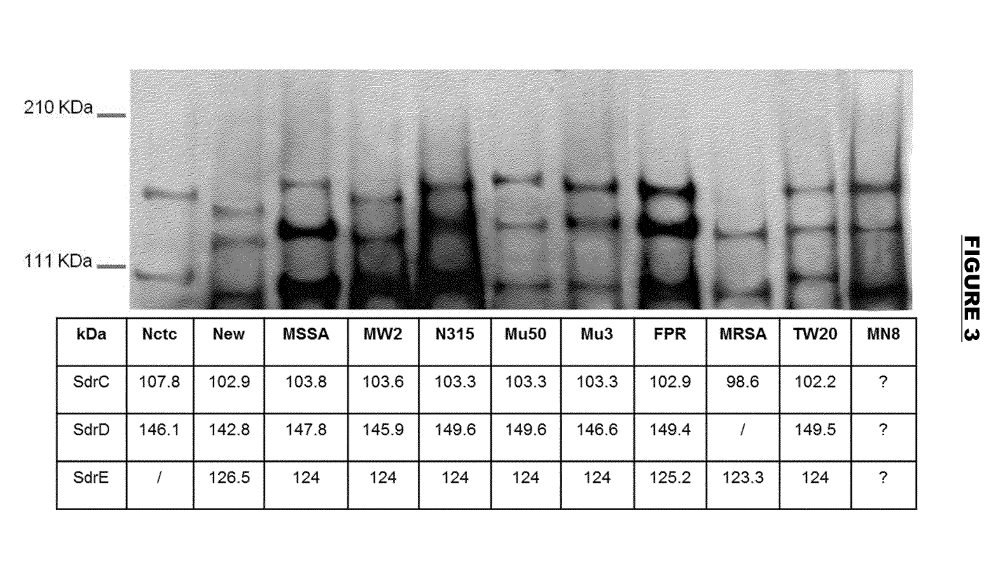

SdrE is not universally expressed by S. aureus strains, so mice immunised with CnaBE3 were tested for protection against a SdrE-negative strain (NCTC8325). Surprisingly, the mice were again protected (see FIG. 2; p=0.017), so CnaBE3 is able to provide cross-protection. This effect could be due to the high sequence identity between CnaB domains (BC2 of SdrC, BD5 of SdrD and BE3 of SdrE) lying adjacent to the ‘R’ region (see FIG. 1 of ref. 3) in these three Sdr proteins of the Newman strain. For instance, anti-CnaBE3 polyclonal serum was incubated with protein extracts from 11 different strains of S. aureus and it recognised proteins with MWs which correspond to each of SdrC, SdrD and SdrE (see FIG. 3). As expected, SdrD was not detected in SdrD^{-ve} strain MRSA252, and SdrE was not detected in SdrE^{-ve} strain NCTC8325. Thus cross-reactivity with SdrC and SdrD could explain the ability of CnaBE3 to protect against a SdrE^{-ve} strain.

### Patient Serum Cross-Reactivity

Sera were obtained from 16 sera healthy neonates (12 to 18 months old), 30 healthy adults (21 to 75 years old), and 30 patients (0 to 81 years old) with proven S. aureus infection as the only microbiological etiology of disease. In addition, healthy adult sera were purchased from 3H Biomedical AB.

These sera were used in an ELISA. Briefly, Nunc MaxiSorp™ flat-bottom 96-well plates were coated (100 μl per well) overnight at 4° C. with 2 μg/ml of rCnaBE3 protein in PBS. The plates were washed three times with TPBS (0.05% (v/v) Tween 20 in PBS, pH 7.4) and blocked with 200 μl per well of Blocking Buffer containing 3% (w/v) BSA (Sigma-Aldrich) in PBS for 2 hr at 37° C. The sera were initially diluted 1:100 in Dilution Buffer (1% (w/v) BSA in TPBS) added in duplicate to the wells (100 μl per well), and serially two-fold diluted. After 2 hr incubation at 37° C., the plates were washed three times with TPBS, then Dilution Buffer containing goat anti-human IgG (λ-chain specific) alkaline phosphatase conjugate affinity isolated antibody (Sigma-Aldrich) diluted 1:2,000 was added 100 μl per well. Following 1 hr and 30 min incubation at 37° C., the plates were washed three times with TPBS, and 100 μl per well of a solution of DEA buffer (1M diethanolamine (v/v), 0.5 mM MgCl₂, 0.02% (w/v) sodium azide, pH 9.8) containing 3 mg/ml p-nitrophenyl phosphate were applied. The reaction was stopped after 20 min by the addition of 100 μl 4N NaOH. Optical densities at 405 nm were measured using SpectraMax 190 Absorbance Microplate Reader supplied with SoftMax™ Pro Data Acquisition & Analysis Software. Antibody titers were calculated by interpolating ODs into the reference calibration curve and expressed in Log Arbitrary Units (lnAU).

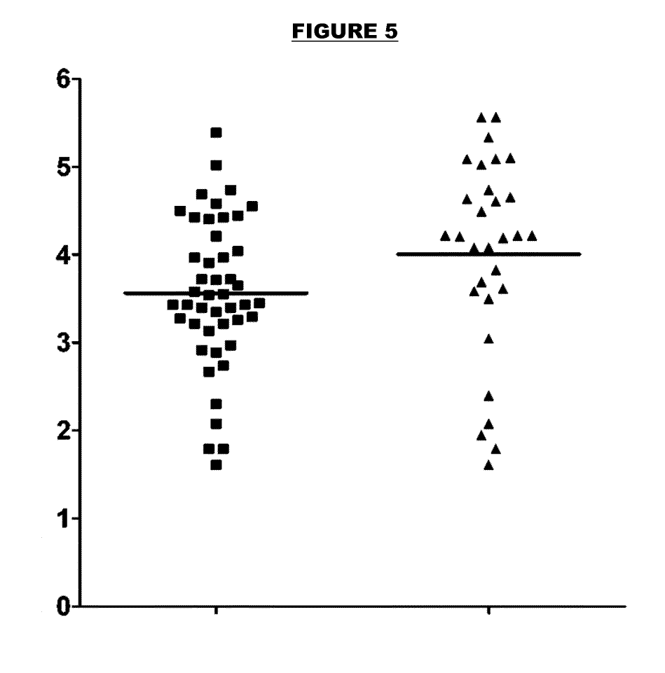

As shown in FIG. 5, the average binding value of the sera to the immobilized CnaBE3 domain was significantly higher for infected patients than with sera from healthy patients (p<0.05). These data suggested that a specific immune response against CnaBE3 fragment was indeed induced during S. aureus infection, indicating that the CnaBE3 domain in SdrE is naturally immunogenic.

### Opsonophagocytosis Killing Assay

Human promyelocytic leukemia cells HL-60 (ATCC CCL240) were maintained in enriched medium and differentiated into phagocytes using 0.8% N,N-dimethylformamide. Following heat inactivation (30 min, 56° C.), mouse CnaBE3 and SdrE antisera were pre-diluted 1:50 in HBSS buffer (with Ca²⁺/Mg²⁺). Bacteria grown overnight in TSB were washed once in PBS, then incubated with serum (75 000 CFU/well) at 4° C. for 20 minutes. Differentiated HL-60 cells were distributed at 3.7×10⁶ per well (HL-60:bacteria ratio, 50:1) and rabbit complement was added at 10% final concentration. Plates were then incubated at 37° C. for 1 hour, under agitation at 600 rpm and samples were plated onto TSA plates for CFU counts determination. Sera were tested at 1:50, 1:500 or 1:5000 dilutions.

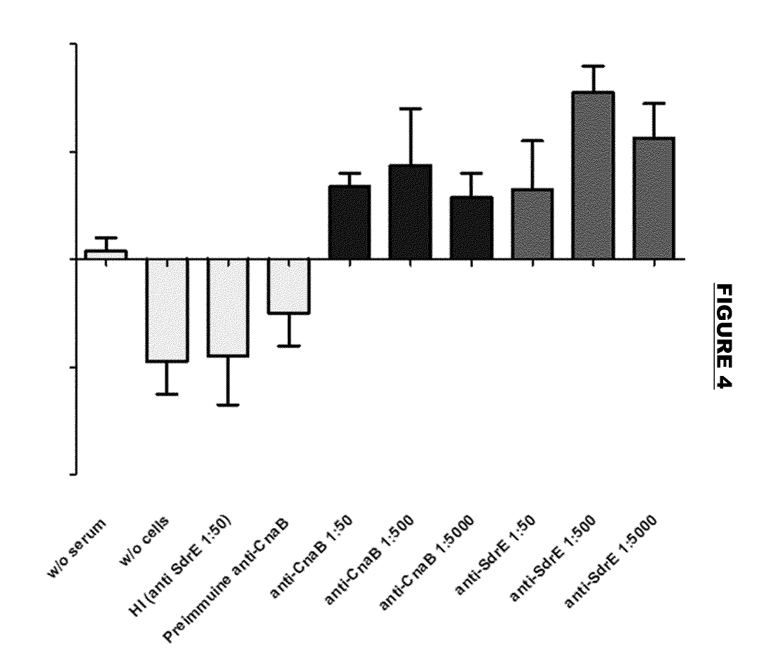

As shown in FIG. 4, when complement was present HL-60 cells killed around 20% of Newman cells in the presence of anti-CnaBE3 serum and around 30% in the presence of anti-SdrE serum, whereas no Newman cells were killed with pre-immune serum.

It will be understood that the invention has been described by way of example only and modifications may be made whilst remaining within the scope and spirit of the invention.

### REFERENCES

[1] WO2010/119343.
[2] WO2010/079464.
[3] Josefsson et al. (1998) Microbiology 144:3387-3395.
[4] Sharp et al. (2012) PLoS ONE 7(5): e38407.
[5] Xiang et al. (2010) Acta Crystallogr Sect F Struct Biol Cryst Commun. 66(Pt 7):858-61.
[6] McCarthy & Lindsay J A (2010) BMC Microbiol. 10:173.
[7] Stranger-Jones et al. (2006) PNAS USA 103(45):16942-7.
[8] O'Brien et al. (2002) Mol Microbiol. 44(4):1033-44.
[9] Deivanayagam et al. (2000) Structure Fold Des 8:67-78.
[10] Andreeva et al. (2004) Nucl. Acid Res. 32:D226-D229.
[11] Finn et al. (2010) Nucl. Acid Res. 38:D211-D222.
[12] Marchler-Bauer A et al. (2011) Nucleic Acids Res. 39:D225-D229.
[13] WO2007/113224.
[14] WO98/10788.
[15] WO2007/053176.
[16] O'Brien et al. (2000) J Dairy Sci 83:1758-66.
[17] Research Disclosure, 453077 (January 2002).
[18] EP-A-0372501.
[19] EP-A-0378881.
[20] EP-A-0427347.
[21] WO93/17712.
[22] WO94/03208.
[23] WO98/58668.
[24] EP-A-0471177.
[25] WO91/01146.
[26] Falugi et al. (2001) Eur J Immunol 31:3816-3824.
[27] Baraldo et al. (2004) Infect Immun 72(8):4884-7.
[28] EP-A-0594610.
[29] Ruan et al. (1990) J Immunol 145:3379-3384.
[30] WO00/56360.
[31] Kuo et al. (1995) Infect Immun 63:2706-13.
[32] Michon et al. (1998) Vaccine. 16:1732-41.
[33] WO02/091998.
[34] WO01/72337.
[35] WO00/61761.
[36] WO00/33882
[37] U.S. Pat. No. 4,761,283.
[38] U.S. Pat. No. 4,356,170.
[39] U.S. Pat. No. 4,882,317.
[40] U.S. Pat. No. 4,695,624.
[41] Mol. Immunol., 1985, 22, 907-919
[42] EP-A-0208375.
[43] Bethell G. S. et al., J. Biol. Chem., 1979, 254, 2572-4
[44] Hearn M. T. W., J. Chromatogr., 1981, 218, 509-18
[45] WO00/10599.
[46] Gever et al., Med. Microbiol. Immunol, 165: 171-288 (1979).
[47] U.S. Pat. No. 4,057,685.
[48] U.S. Pat. Nos. 4,673,574; 4,761,283; 4,808,700.
[49] U.S. Pat. No. 4,459,286.
[50] U.S. Pat. No. 4,965,338.
[51] U.S. Pat. No. 4,663,160.
[52] WO2007/000343.
[53] Rable & Wardenburg (2009) Infect Immun 77:2712-8.
[54] WO2007/145689.
[55] WO2009/029831.
[56] Sebulsky & Heinrichs (2001) J Bacteriol 183:4994-5000.
[57] Sebulsky et al. (2003) J Biol Chem 278:49890-900.
[58] WO2008/019162.
[59] Kuroda et cd. (2001) Lancet 357:1225-1240.
[60] Needleman & Wunsch (1970) J. Mol. Biol. 48, 443-453.
[61] Rice et al. (2000) Trends Genet 16:276-277.
[62] Remington: The Science and Practice of Pharmacy (Gennaro, 2000; 20th edition, ISBN: 0683306472)
[63] WO2011/067669.
[64] WO2011/067672.
[65] WO2011/067673.
[66] WO2008/056263.
[67] WO2011/154442.
[68] WO2011/154443.
[69] WO2011/154444.
[70] WO90/14837.
[71] Podda & Del Giudice (2003) Expert Rev Vaccines 2:197-203.
[72] Podda (2001) Vaccine 19: 2673-2680.
[73] WO2008/043774.
[74] WO2005/097181.
[75] WO95/11700.
[76] US-2007/014805.
[77] WO2007/080308.
[78] WO2010/023551
[79] Brito et al. (2011) Vaccine 29:6262-6268.
[80] U.S. Pat. No. 8,092,813.
[81] WO2011/141819.
[82] U.S. Pat. No. 6,630,161.
[83] Vaccine Design: The Subunit and Adjuvant Approach (eds. Powell & Newman) Plenum Press 1995 (ISBN 0-306-44867-X).
[84] Vaccine Adjuvants: Preparation Methods and Research Protocols (Volume 42 of Methods in Molecular Medicine series). ISBN: 1-59259-083-7. Ed. O'Hagan.
[85] Clausi et al. (2008) J Pharm Sci DOI 10.1002/jps.21390.
[86] WO2012/031140.
[87] Stranger-Jones et al. (2006) PNAS USA 103:16942-7.
[88] Wardenburg et al. (2007) Infect Immun 75:1040-4.
[89] Donnelly et al. (1997) Annu Rev Immunol 15:617-648.
[90] Strugnell et al. (1997) Immunol Cell Biol 75(4):364-369.
[91] Cui (2005) Adv Genet 54:257-89.
[92] Robinson & Torres (1997) Seminars in Immunol 9:271-283.
[93] Brunham et al. (2000) J Infect Dis 181 Suppl 3:S538-43.
[94] Svanholm et al. (2000) Scand J Immunol 51(4):345-53.
[95] DNA Vaccination—Genetic Vaccination (1998) eds. Koprowski et al. (ISBN 3540633928).
[96] Gene Vaccination: Theory and Practice (1998) ed. Raz (ISBN 3540644288).
[97] Corrigan et al. (2009) BMC Microbiol 9:22.

1. A polypeptide comprising a SdrE CnaBE3 domain, wherein the polypeptide does not comprise (i) a full-length SdrE protein or (ii) an amino acid sequence of formula (SEQ ID NO: 5)-X¹-(SEQ ID NO: 3)-X²-(SEQ ID NO: 6), where X¹ is an optional PSTSE (SEQ ID NO:44) sequence and X² is between 20-250 amino acids long and is either (i) multiple repeats of SD or (ii) a mixture of both SD and AD sequences.

2. The polypeptide of claim 1 comprising a fragment of a S. aureus SdrE protein, wherein: (a) the fragment includes the SdrE's CnaBE3 domain; (b) the polypeptide does not comprise a full-length SdrE protein; and (c) the polypeptide does not comprise an amino acid sequence of formula (SEQ ID NO: 5)-X¹-(SEQ ID NO: 3)-X²-(SEQ ID NO: 6), where X¹ is an optional PSTSE (SEQ ID NO:44) sequence and X² is between 20-250 amino acids long and is either (i) multiple repeats of SD or (ii) a mixture of both SD and AD sequences.

3. The polypeptide of claim 2, wherein the SdrE protein has ≧90% identity to SEQ ID NO: 3.

4. The polypeptide of claim 1 wherein the CnaBE3 domain has at least 95% identity to SEQ ID NO: 8.

5. The polypeptide of claim 1, comprising SEQ ID NO: 8 or SEQ ID NO: 27.

6. The polypeptide of claim 1, wherein the polypeptide, when administered to a human or mouse, elicits antibodies which recognise an epitope within SEQ ID NO: 8 or within SEQ ID NO: 27.

7. The polypeptide of claim 1, wherein the polypeptide has <500 amino acids.

8. A polypeptide comprising a mutant S. aureus SdrE CnaBE3 domain wherein:
at one or more amino acid position(s) where the native S. aureus SdrE CnaBE3 domain has an asparagine residue, the mutant has either (i) an amino acid deletion or (ii) an amino acid substitution;
and/or
at one or more amino acid position(s) where the native S. aureus SdrE CnaBE3 domain has an aspartate residue, the mutant has either (i) an amino acid deletion or (ii) an amino acid substitution;
and/or
at one or more amino acid position(s) where the native S. aureus SdrE CnaBE3 domain has a lysine residue, the mutant has either (i) an amino acid deletion or (ii) an amino acid substitution.

9. The polypeptide of claim 8, comprising the amino acid sequence of any one of SEQ ID NOs: 9 to 26.

10. A polypeptide comprising a S. aureus CnaB domain, wherein the CnaB domain includes an isopeptide bond.

11. The polypeptide of claim 10, wherein the S. aureus CnaB domain is from S. aureus SdrE.

12. The polypeptide of claim 11, wherein the S. aureus CnaB domain is the CnaBE3 domain.

13. A polypeptide comprising at least two CnaB domains, wherein: either (a) at least one of the CnaB domains is a SdrE CnaBE3 domain and at least one CnaB domain is not a SdrE CnaB domain; or (b) the polypeptide comprises at least two SdrE CnaBE3 domains.

14. An immunogenic composition comprising the polypeptide of claim 1.

15. The composition of claim 14, further comprising one or more of: (a) a conjugate of a S. aureus exopolysaccharide and a carrier protein; (b) a conjugate of a S. aureus capsular saccharide and a carrier protein; (c) a S. aureus polypeptide other than a SdrE polypeptide; and/or (d) a non-staphylococcal antigen.

16. The composition of claim 15, including an immunological adjuvant.

17. A method for raising an immune response in a mammal comprising the step of administering an effective amount of the immunogenic composition of claim 14.

18. Nucleic acid encoding the polypeptide of claim 1.

19. The method of claim 17, wherein the mammal is a human.

20. The composition of claim 14, further comprising a TLR agonist.


SEQUENCE LISTING (46 sequences)

SEQ ID NO: 1 - protein, 1166 aa, Staphylococcus aureus
MINRDNKKAITKKGMISNRLNKFSIRKYTVGTASILVGTTLIFGLGNQEAKAAENTSTENAKQDDATTSDNKEVVSETENNSTTENNSTNPIKKETNTDSQPEAKKESTSSSTQKQQNNVTATTETKPQNIEKENVKPSTDKTATEDTSVILEEKKAPNNTNNDVTTKPSTSEPSTSEIQTKPTTPQESTNIENSQPQPTPSKVDNQVTDATNPKEPVNVSKEELKNNPEKLKELVRNDSNTDHSTKPVATAPTSVAPKRVNAKMRFAVAQPAAVASNNVNDLIKVTKQTIKVGDGKDNVAAAHDGKDIEYDTEFTIDNKVKKGDTMTINYDKNVIPSDLTDKNDPIDITDPSGEVIAKGTFDKATKQITYTFTDYVDKYEDIKSRLTLYSYIDKKTVPNETSLNLTFATAGKETSQNVTVDYQDPMVHGDSNIQSIFTKLDEDKQTIEQQIYVNPLKKSATNTKVDIAGSQVDDYGNIKLGNGSTIIDQNTEIKVYKVNSDQQLPQSNRIYDFSQYEDVTSQFDNKKSFSNNVATLDFGDINSAYIIKVVSKYTPTSDGELDIAQGTSMRTTDKYGYYNYAGYSNFIVTSNDTGGGDGTVKPEEKLYKIGDYVWEDVDKDGVQGTDSKEKPMANVLVTLTYPDGTTKSVRTDANGHYEFGGLKDGETYTVKFETPTGYLPTKVNGTTDGEKDSNGSSVTVKINGKDDMSLDTGFYKEPKYNLGDYVWEDTNKDGIQDANEPGIKDVKVTLKDSTGKVIGTTTTDASGKYKFTDLDNGNYTVEFETPAGYTPTVKNTTADDKDSNGLTTTGVIKDADNMTLDRGFYKTPKYSLGDYVWYDSNKDGKQDSTEKGIKDVTVTLQNEKGEVIGTTKTDENGKYRFDNLDSGKYKVIFEKPAGLTQTVTNTTEDDKDADGGEVDVTITDHDDFTLDNGYFEEDTSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDAGKHTPVKPMSTTKDHHNKAKALPETGSENNGSNNATLFGGLFAALGSLLLFGRRKKQNK

SEQ ID NO: 2 - protein, 168 aa, Staphylococcus aureus
MINRDNKKAITKKGMISNRLNKFSIRKYTVGTASILVGTTLIFGLGNQEAKAAENTSTENAKQDDATTSDNKEVVSETENNSTTENNSTNPIKKETNTDSQPEAKKESTSSSTQKQQNNVTATTETKPQNIEKENVKPSTDKTATEDTSVILEEKKAPNNTNNDVTTK

SEQ ID NO: 3 - protein, 767 aa, Staphylococcus aureus
PSTSEIQTKPTTPQESTNIENSQPQPTPSKVDNQVTDATNPKEPVNVSKEELKNNPEKLKELVRNDSNTDHSTKPVATAPTSVAPKRVNAKMRFAVAQPAAVASNNVNDLIKVTKQTIKVGDGKDNVAAAHDGKDIEYDTEFTIDNKVKKGDTMTINYDKNVIPSDLTDKNDPIDITDPSGEVIAKGTFDKATKQITYTFTDYVDKYEDIKSRLTLYSYIDKKTVPNETSLNLTFATAGKETSQNVTVDYQDPMVHGDSNIQSIFTKLDEDKQTIEQQIYVNPLKKSATNTKVDIAGSQVDDYGNIKLGNGSTIIDQNTEIKVYKVNSDQQLPQSNRIYDFSQYEDVTSQFDNKKSFSNNVATLDFGDINSAYIIKVVSKYTPTSDGELDIAQGTSMRTTDKYGYYNYAGYSNFIVTSNDTGGGDGTVKPEEKLYKIGDYVWEDVDKDGVQGTDSKEKPMANVLVTLTYPDGTTKSVRTDANGHYEFGGLKDGETYTVKFETPTGYLPTKVNGTTDGEKDSNGSSVTVKINGKDDMSLDTGFYKEPKYNLGDYVWEDTNKDGIQDANEPGIKDVKVTLKDSTGKVIGTTTTDASGKYKFTDLDNGNYTVEFETPAGYTPTVKNTTADDKDSNGLTTTGVIKDADNMTLDRGFYKTPKYSLGDYVWYDSNKDGKQDSTEKGIKDVTVTLQNEKGEVIGTTKTDENGKYRFDNLDSGKYKVIFEKPAGLTQTVTNTTEDDKDADGGEVDVTITDHDDFTLDNGYFEEDT

SEQ ID NO: 4 - protein, 60 aa, Staphylococcus aureus
AGKHTPVKPMSTTKDHHNKAKALPETGSENNGSNNATLFGGLFAALGSLLLFGRRKKQNK

SEQ ID NO: 5 - protein, 116 aa, Staphylococcus aureus
AENTSTENAKQDDATTSDNKEVVSETENNSTTENNSTNPIKKETNTDSQPEAKKESTSSSTQKQQNNVTATTETKPQNIEKENVKPSTDKTATEDTSVILEEKKAPNNTNNDVTTK

SEQ ID NO: 6 - protein, 22 aa, Staphylococcus aureus
AGKHTPVKPMSTTKDHHNKAKA

SEQ ID NO: 7 - protein, 1076 aa, Staphylococcus aureus
AENTSTENAKQDDATTSDNKEVVSETENNSTTENNSTNPIKKETNTDSQPEAKKESTSSSTQKQQNNVTATTETKPQNIEKENVKPSTDKTATEDTSVILEEKKAPNNTNNDVTTKPSTSEPSTSEIQTKPTTPQESTNIENSQPQPTPSKVDNQVTDATNPKEPVNVSKEELKNNPEKLKELVRNDSNTDHSTKPVATAPTSVAPKRVNAKMRFAVAQPAAVASNNVNDLIKVTKQTIKVGDGKDNVAAAHDGKDIEYDTEFTIDNKVKKGDTMTINYDKNVIPSDLTDKNDPIDITDPSGEVIAKGTFDKATKQITYTFTDYVDKYEDIKSRLTLYSYIDKKTVPNETSLNLTFATAGKETSQNVTVDYQDPMVHGDSNIQSIFTKLDEDKQTIEQQIYVNPLKKSATNTKVDIAGSQVDDYGNIKLGNGSTIIDQNTEIKVYKVNSDQQLPQSNRIYDFSQYEDVTSQFDNKKSFSNNVATLDFGDINSAYIIKVVSKYTPTSDGELDIAQGTSMRTTDKYGYYNYAGYSNFIVTSNDTGGGDGTVKPEEKLYKIGDYVWEDVDKDGVQGTDSKEKPMANVLVTLTYPDGTTKSVRTDANGHYEFGGLKDGETYTVKFETPTGYLPTKVNGTTDGEKDSNGSSVTVKINGKDDMSLDTGFYKEPKYNLGDYVWEDTNKDGIQDANEPGIKDVKVTLKDSTGKVIGTTTTDASGKYKFTDLDNGNYTVEFETPAGYTPTVKNTTADDKDSNGLTTTGVIKDADNMTLDRGFYKTPKYSLGDYVWYDSNKDGKQDSTEKGIKDVTVTLQNEKGEVIGTTKTDENGKYRFDNLDSGKYKVIFEKPAGLTQTVTNTTEDDKDADGGEVDVTITDHDDFTLDNGYFEEDTSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDAGKHTPVKPMSTTKDHHNKAKA

SEQ ID NO: 8 - protein, 111 aa, Staphylococcus aureus
KYSLGDYVWYDSNKDGKQDSTEKGIKDVTVTLQNEKGEVIGTTKTDENGKYRFDNLDSGKYKVIFEKPAGLTQTVTNTTEDDKDADGGEVDVTITDHDDFTLDNGYFEEDT

SEQ ID NO: 9 - protein, 111 aa, Staphylococcus aureus
  MOD_RES (13)..(13): Any amino acid except Asn
KYSLGDYVWYDSXKDGKQDSTEKGIKDVTVTLQNEKGEVIGTTKTDENGKYRFDNLDSGKYKVIFEKPAGLTQTVTNTTEDDKDADGGEVDVTITDHDDFTLDNGYFEEDT

SEQ ID NO: 10 - protein, 111 aa, Staphylococcus aureus
  MOD_RES (34)..(34): Any amino acid except Asn
KYSLGDYVWYDSNKDGKQDSTEKGIKDVTVTLQXEKGEVIGTTKTDENGKYRFDNLDSGKYKVIFEKPAGLTQTVTNTTEDDKDADGGEVDVTITDHDDFTLDNGYFEEDT

SEQ ID NO: 11 - protein, 111 aa, Staphylococcus aureus
  MOD_RES (48)..(48): Any amino acid except Asn
KYSLGDYVWYDSNKDGKQDSTEKGIKDVTVTLQNEKGEVIGTTKTDEXGKYRFDNLDSGKYKVIFEKPAGLTQTVTNTTEDDKDADGGEVDVTITDHDDFTLDNGYFEEDT

SEQ ID NO: 12 - protein, 111 aa, Staphylococcus aureus
  MOD_RES (55)..(55): Any amino acid except Asn
KYSLGDYVWYDSNKDGKQDSTEKGIKDVTVTLQNEKGEVIGTTKTDENGKYRFDXLDSGKYKVIFEKPAGLTQTVTNTTEDDKDADGGEVDVTITDHDDFTLDNGYFEEDT

SEQ ID NO: 13 - protein, 111 aa, Staphylococcus aureus
  MOD_RES (77)..(77): Any amino acid except Asn
KYSLGDYVWYDSNKDGKQDSTEKGIKDVTVTLQNEKGEVIGTTKTDENGKYRFDNLDSGKYKVIFEKPAGLTQTVTXTTEDDKDADGGEVDVTITDHDDFTLDNGYFEEDT

SEQ ID NO: 14 - protein, 111 aa, Staphylococcus aureus
  MOD_RES (104)..(104): Any amino acid except Asn
KYSLGDYVWYDSNKDGKQDSTEKGIKDVTVTLQNEKGEVIGTTKTDENGKYRFDNLDSGKYKVIFEKPAGLTQTVTNTTEDDKDADGGEVDVTITDHDDFTLDXGYFEEDT

SEQ ID NO: 15 - protein, 111 aa, Staphylococcus aureus
  MOD_RES (1)..(1): Any amino acid except Lys
XYSLGDYVWYDSNKDGKQDSTEKGIKDVTVTLQNEKGEVIGTTKTDENGKYRFDNLDSGKYKVIFEKPAGLTQTVTNTTEDDKDADGGEVDVTITDHDDFTLDNGYFEEDT

SEQ ID NO: 16 - protein, 111 aa, Staphylococcus aureus
  MOD_RES (14)..(14): Any amino acid except Lys
KYSLGDYVWYDSNXDGKQDSTEKGIKDVTVTLQNEKGEVIGTTKTDENGKYRFDNLDSGKYKVIFEKPAGLTQTVTNTTEDDKDADGGEVDVTITDHDDFTLDNGYFEEDT

SEQ ID NO: 17 - protein, 111 aa, Staphylococcus aureus
  MOD_RES (17)..(17): Any amino acid except Lys
KYSLGDYVWYDSNKDGXQDSTEKGIKDVTVTLQNEKGEVIGTTKTDENGKYRFDNLDSGKYKVIFEKPAGLTQTVTNTTEDDKDADGGEVDVTITDHDDFTLDNGYFEEDT

SEQ ID NO: 18 - protein, 111 aa, Staphylococcus aureus
  MOD_RES (23)..(23): Any amino acid except Lys
KYSLGDYVWYDSNKDGKQDSTEXGIKDVTVTLQNEKGEVIGTTKTDENGKYRFDNLDSGKYKVIFEKPAGLTQTVTNTTEDDKDADGGEVDVTITDHDDFTLDNGYFEEDT

SEQ ID NO: 19 - protein, 111 aa, Staphylococcus aureus
  MOD_RES (26)..(26): Any amino acid except Lys
KYSLGDYVWYDSNKDGKQDSTEKGIXDVTVTLQNEKGEVIGTTKTDENGKYRFDNLDSGKYKVIFEKPAGLTQTVTNTTEDDKDADGGEVDVTITDHDDFTLDNGYFEEDT

SEQ ID NO: 20 - protein, 111 aa, Staphylococcus aureus
  MOD_RES (36)..(36): Any amino acid except Lys
KYSLGDYVWYDSNKDGKQDSTEKGIKDVTVTLQNEXGEVIGTTKTDENGKYRFDNLDSGKYKVIFEKPAGLTQTVTNTTEDDKDADGGEVDVTITDHDDFTLDNGYFEEDT

SEQ ID NO: 21 - protein, 111 aa, Staphylococcus aureus
  MOD_RES (44)..(44): Any amino acid except Lys
KYSLGDYVWYDSNKDGKQDSTEKGIKDVTVTLQNEKGEVIGTTXTDENGKYRFDNLDSGKYKVIFEKPAGLTQTVTNTTEDDKDADGGEVDVTITDHDDFTLDNGYFEEDT

SEQ ID NO: 22 - protein, 111 aa, Staphylococcus aureus
  MOD_RES (50)..(50): Any amino acid except Lys
KYSLGDYVWYDSNKDGKQDSTEKGIKDVTVTLQNEKGEVIGTTKTDENGXYRFDNLDSGKYKVIFEKPAGLTQTVTNTTEDDKDADGGEVDVTITDHDDFTLDNGYFEEDT

SEQ ID NO: 23 - protein, 111 aa, Staphylococcus aureus
  MOD_RES (60)..(60): Any amino acid except Lys
KYSLGDYVWYDSNKDGKQDSTEKGIKDVTVTLQNEKGEVIGTTKTDENGKYRFDNLDSGXYKVIFEKPAGLTQTVTNTTEDDKDADGGEVDVTITDHDDFTLDNGYFEEDT

SEQ ID NO: 24 - protein, 111 aa, Staphylococcus aureus
  MOD_RES (62)..(62): Any amino acid except Lys
KYSLGDYVWYDSNKDGKQDSTEKGIKDVTVTLQNEKGEVIGTTKTDENGKYRFDNLDSGKYXVIFEKPAGLTQTVTNTTEDDKDADGGEVDVTITDHDDFTLDNGYFEEDT

SEQ ID NO: 25 - protein, 111 aa, Staphylococcus aureus
  MOD_RES (67)..(67): Any amino acid except Lys
KYSLGDYVWYDSNKDGKQDSTEKGIKDVTVTLQNEKGEVIGTTKTDENGKYRFDNLDSGKYKVIFEXPAGLTQTVTNTTEDDKDADGGEVDVTITDHDDFTLDNGYFEEDT

SEQ ID NO: 26 - protein, 111 aa, Staphylococcus aureus
  MOD_RES (83)..(83): Any amino acid except Lys
KYSLGDYVWYDSNKDGKQDSTEKGIKDVTVTLQNEKGEVIGTTKTDENGKYRFDNLDSGKYKVIFEKPAGLTQTVTNTTEDDXDADGGEVDVTITDHDDFTLDNGYFEEDT

SEQ ID NO: 27 - protein, 126 aa, Staphylococcus aureus
DADNMTLDRGFYKTPKYSLGDYVWYDSNKDGKQDSTEKGIKDVTVTLQNEKGEVIGTTKTDENGKYRFDNLDSGKYKVIFEKPAGLTQTVTNTTEDDKDADGGEVDVTITDHDDFTLDNGYFEEDT

SEQ ID NO: 28 - protein, 319 aa, Staphylococcus aureus
MKTRIVSSVTTTLLLGSILMNPVANAADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 29 - protein, 293 aa, Staphylococcus aureus
ADSDINIKTGTTDIGSNTTVKTGDLVTYDKENGMLKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 30 - protein, 296 aa, Staphylococcus aureus
MASADSDINIKTGTTDIGSNTTVKTGDLVTYDKENGMLKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 31 - protein, 302 aa, Staphylococcus aureus
MKKLLLPLIIMLLVLAACGNQGEKNNKAETKSYKMDDGKTVDIPKDPKRIAVVAPTYAGGLKKLGANIVAVNQQVDQSKVLKDKFKGVTKIGDGDVEKVAKEKPDLIIVYSTDKDIKKYQKVAPTVVVDYNKHKYLEQQEMLGKIVGKEDKVKAWKKDWEETTAKDGKEIKKAIGQDATVSLFDEFDKKLYTYGDNWGRGGEVLYQAFGLKMQPEQQKLTAKAGWAEVKQEEIEKYAGDYIVSTSEGKPTPGYESTNMWKNLKATKEGHIVKVDAGTYWYNDPYTLDFMRKDLKEKLIKAAK

SEQ ID NO: 32 - protein, 288 aa, Staphylococcus aureus
MASCGNQGEKNNKAETKSYKMDDGKTVDIPKDPKRIAVVAPTYAGGLKKLGANIVAVNQQVDQSKVLKDKFKGVTKIGDGDVEKVAKEKPDLIIVYSTDKDIKKYQKVAPTVVVDYNKHKYLEQQEMLGKIVGKEDKVKAWKKDWEETTAKDGKEIKKAIGQDATVSLFDEFDKKLYTYGDNWGRGGEVLYQAFGLKMQPEQQKLTAKAGWAEVKQEEIEKYAGDYIVSTSEGKPTPGYESTNMWKNLKATKEGHIVKVDAGTYWYNDPYTLDFMRKDLKEKLIKAAK

SEQ ID NO: 33 - protein, 287 aa, Staphylococcus aureus
MASGNQGEKNNKAETKSYKMDDGKTVDIPKDPKRIAVVAPTYAGGLKKLGANIVAVNQQVDQSKVLKDKFKGVTKIGDGDVEKVAKEKPDLIIVYSTDKDIKKYQKVAPTVVVDYNKHKYLEQQEMLGKIVGKEDKVKAWKKDWEETTAKDGKEIKKAIGQDATVSLFDEFDKKLYTYGDNWGRGGEVLYQAFGLKMQPEQQKLTAKAGWAEVKQEEIEKYAGDYIVSTSEGKPTPGYESTNMWKNLKATKEGHIVKVDAGTYWYNDPYTLDFMRKDLKEKLIKAAK

SEQ ID NO: 34 - protein, 256 aa, Staphylococcus aureus
MMKRLNKLVLGIIFLFLVISITAGCGIGKEAEVKKSFEKTLSMYPIKNLEDLYDKEGYRDDQFDKNDKGTWIINSEMVIQPNNEDMVAKGMVLYMNRNTKTTNGYYYVDVTKDEDEGKPHDNEKRYPVKMVDNKIIPTKEIKDEKIKKEIENFKFFVQYGDFKNLKNYKDGDISYNPEVPSYSAKYQLTNDDYNVKQLRKRYDIPTSKAPKLLLKGSGNLKGSSVGYKDIEFTFVEKKEENIYFSDSLDYKKSGDV

SEQ ID NO: 35 - protein, 234 aa, Staphylococcus aureus
MGCGIGKEAEVKKSFEKTLSMYPIKNLEDLYDKEGYRDDQFDKNDKGTWIINSEMVIQPNNEDMVAKGMVLYMNRNTKTTNGYYYVDVTKDEDEGKPHDNEKRYPVKMVDNKIIPTKEIKDEKIKKEIENFKFFVQYGDFKNLKNYKDGDISYNPEVPSYSAKYQLTNDDYNVKQLRKRYDIPTSKAPKLLLKGSGNLKGSSVGYKDIEFTFVEKKEENIYFSDSLDYKKSGDV

SEQ ID NO: 36 - protein, 234 aa, Staphylococcus aureus
MGSGIGKEAEVKKSFEKTLSMYPIKNLEDLYDKEGYRDDQFDKNDKGTWIINSEMVIQPNNEDMVAKGMVLYMNRNTKTTNGYYYVDVTKDEDEGKPHDNEKRYPVKMVDNKIIPTKEIKDEKIKKEIENFKFFVQYGDFKNLKNYKDGDISYNPEVPSYSAKYQLTNDDYNVKQLRKRYDIPTSKAPKLLLKGSGNLKGSSVGYKDIEFTFVEKKEENIYFSDSLDYKKSGDV

SEQ ID NO: 37 - protein, 97 aa, Staphylococcus aureus
MAMIKMSPEEIRAKSQSYGQGSDQIRQILSDLTRAQGEIAANWEGQAFSRFEEQFQQLSPKVEKFAQLLEEIKQQLNSTADAVQEQDQQLSNNFGLQ

SEQ ID NO: 38 - protein, 104 aa, Staphylococcus aureus
MGGYKGIKADGGKVDQAKQLAAKTAKDIEACQKQTQQLAEYIEGSDWEGQFANKVKDVLLIMAKFQEELVQPMADHQKAIDNLSQNLAKYDTLSIKQGLDRVNP

SEQ ID NO: 39 - protein, 207 aa, Staphylococcus aureus
MAMIKMSPEEIRAKSQSYGQGSDQIRQILSDLTRAQGEIAANWEGQAFSRFEEQFQQLSPKVEKFAQLLEEIKQQLNSTADAVQEQDQQLSNNFGLQASGGGSMGGYKGIKADGGKVDQAKQLAAKTAKDIEACQKQTQQLAEYIEGSDWEGQFANKVKDVLLIMAKFQEELVQPMADHQKAIDNLSQNLAKYDTLSIKQGLDRVNP

SEQ ID NO: 40 - protein, 6 aa, Artificial Sequence
  source: /note=“Description of Artificial Sequence Synthetic peptide”
ASGGGS

SEQ ID NO: 41 - protein, 205 aa, Artificial Sequence
  source: /note=“Description of Artificial Sequence Synthetic polypeptide”
AMIKMSPEEIRAKSQSYGQGSDQIRQILSDLTRAQGEIAANWEGQAFSRFEEQFQQLSPKVEKFAQLLEEIKQQLNSTADAVQEQDQQLSNNFGLQASGGGSGGYKGIKADGGKVDQAKQLAAKTAKDIEACQKQTQQLAEYIEGSDWEGQFANKVKDVLLIMAKFQEELVQPMADHQKAIDNLSQNLAKYDTLSIKQGLDRVNP

SEQ ID NO: 42 - protein, 206 aa, Artificial Sequence
  source: /note=“Description of Artificial Sequence Synthetic polypeptide”
MAMIKMSPEEIRAKSQSYGQGSDQIRQILSDLTRAQGEIAANWEGQAFSRFEEQFQQLSPKVEKFAQLLEEIKQQLNSTADAVQEQDQQLSNNFGLQASGGGSGGYKGIKADGGKVDQAKQLAAKTAKDIEACQKQTQQLAEYIEGSDWEGQFANKVKDVLLIMAKFQEELVQPMADHQKAIDNLSQNLAKYDTLSIKQGLDRVNP

SEQ ID NO: 43 - protein, 206 aa, Artificial Sequence
  source: /note=“Description of Artificial Sequence Synthetic polypeptide”
MAMIKMSPEEIRAKSQSYGQGSDQIRQILSDLTRAQGEIAANWEGQAFSRFEEQFQQLSPKVEKFAQLLEEIKQQLNSTADAVQEQDQQLSNNFGLQASGGGSGGYKGIKADGGKVDQAKQLAAKTAKDIEAAQKQTQQLAEYIEGSDWEGQFANKVKDVLLIMAKFQEELVQPMADHQKAIDNLSQNLAKYDTLSIKQGLDRVNP

SEQ ID NO: 44 - protein, 5 aa, Staphylococcus aureus
PSTSE

SEQ ID NO: 45 - protein, 10 aa, Artificial Sequence
  source: /note=“Description of Artificial Sequence Synthetic His tag”
HHHHHHHHHH

SEQ ID NO: 46 - protein, 6 aa, Artificial Sequence
  source: /note=“Description of Artificial Sequence Synthetic 6xHis tag”
HHHHHH